(19) United States
(12) Patent Application Publication
(10) Pub. No.: US 2003/0104531 A1
(43) Pub. Date: Jun. 5, 2003

(54) DNA molecules encoding human nuclear receptor protein nNR7-1

(75) Inventor: Fang Chen, North Wales, PA (US)

Correspondence Address: MERCK AND CO INC, P O BOX 2000, RAHWAY, NJ, 070650907

(73) Assignee: Merck & co., Inc., Rahway, NJ (US)

(21) Appl. No.: 10/090,090

(22) Filed: Mar. 4, 2002

Related U.S. Application Data
(63) Continuation of application No. 09/209,069, filed on Dec. 10, 1998, now abandoned.
(60) Provisional application No. 60/104,251, filed on Oct. 14, 1998.
(60) Provisional application No. 60/069,401, filed on Dec. 12, 1997.

(51) Int. Cl. C07K 14/705; C07H 21/04; C12P 21/02; C12N 5/06

(52) U.S. Cl. 435/69.1; 435/320.1; 435/325; 530/350; 536/23.5

(57) ABSTRACT

The present invention discloses the isolation and characterization of cDNA molecules encoding a novel member to the human nuclear receptor superfamiliy, designated nNR7 and/or nNR7-1. Also within the scope of the disclosure are recombinant vectors, recombinant host cells, methods of screening for modulators of nNR7 and/or nNR7-1 activity, and production of antibodies against nNR7 and/or nNR7-1, or epitopes thereof.

### CROSS-REFERENCE TO RELATED APPLICATIONS

Provisional application No. 60/104,251 filed Oct. 14, 1998, which is a continuation of Provisional application No. 60/069,401 filed Dec. 12, 1997.

### STATEMENT REGARDING FEDERALLY-SPONSORED R&D

Not applicable.

### REFERENCE TO MICROFICHE APPENDIX

Not applicable.

### FIELD OF THE INVENTION

The present invention relates in part to isolated nucleic acid molecules (polynucleotide) which encodes a human nuclear receptor proteins, referred to throughout as nNR7 and nNR7-1, respectively. The present invention also relates to recombinant vectors and recombinant hosts which contain a DNA fragment encoding nNR7 and nNR7-1, substantially purified forms of associated human nNR7 protein and associated human nNR7-1 protein, human mutant proteins of nNR7 and nNR7-1, and methods associated with identifying compounds which modulate nNR7 and nNR7-1 activity.

### BACKGROUND OF THE INVENTION

The nuclear receptor superfamily, which includes steroid hormone receptors, are small chemical ligand-inducible transcription factors which have been shown to play roles in controlling development, differentiation and physiological function. Isolation of cDNA clones encoding nuclear receptors reveal several characteristics. First, the NH₂-terminal regions, which vary in length between receptors, is hypervariable with low homology between family members. There are three internal regions of conservation, referred to as domain I, II and III. Region I is a cysteine-rich region which is referred to as the DNA binding domain (DBD). Regions II and III are within the COOH-terminal region of the protein and is also referred to as the ligand binding domain (LBD). For a review, see Power et al. (1992, Trends in Pharmaceutical Sciences 13: 318-323).

The lipophilic hormones that activate steroid receptors are known to be associated with human diseases. Therefore, the respective nuclear receptors have been identified as possible targets for therapeutic intervention. For a review of the mechanism of action of various steroid hormone receptors, see Tsai and O'Malley (1994, Annu. Rev. Biochem. 63:451-486).

Recent work with non-steroid nuclear receptors has also shown the potential as drug targets for therapeutic intervention. This work reports that peroxisome proliferator activated receptor g (PPARg), identified by a conserved DBD region, promotes adipocyte differentiation upon activation and that thiazolidinediones, a class of antidiabetic drugs, function through PPARg (Tontonoz et al., 1994, Cell 79: 1147-1156; Lehmann et al., 1995, J. Biol. Chem. 270(22): 12953-12956; Teboul et al., 1995, J. Biol. Chem. 270(47): 28183-28187). This indicates that PPARg plays a role in glucose homeostasis and lipid metabolism.

Baes et al. (1994, Mol. Cell. Biol. 14(3):1544-1552) disclose a cDNA clone which encodes a new member of the nuclear receptor superfamily, referred herein as hONX. The authors present data showing that the gene is expressed mainly in human liver and suggests that this nuclear receptor may play a role in regulating expression of retinoid-responsive genes.

Baker, et al. (1988, Proc. Natl. Acad. Sci. (U.S.A) 85 (10): 3294-3298) disclose a cDNA clone which encodies human vitamin D receptor (hVDR).

Mangelsdorf et al. (1995, Cell 83: 835-839) provide a review of known members of the nuclear receptor superfamily, including hONX and hVDR.

It would be advantageous to identify additional genes which are members of the nuclear receptor superfamily, especially vertebrate members from such species as human, rat and mouse. A nucleic acid molecule expressing a nuclear receptor protein will be useful in screening for compounds acting as a modulator of cell differentiation, cell development and physiological function. The present invention addresses and meets these needs by disclosing isolated nucleic acid molecules which express a human nuclear receptor protein which will have a role in cell differentiation and development.

### SUMMARY OF THE INVENTION

The present invention relates to isolated nucleic acid molecules (polynucleotides) which encode novel nuclear receptor proteins which are herein designated as members of the nuclear receptor superfamily. The isolated polynucleotides of the present invention encode vertebrate members of this nuclear receptor superfamily, and preferably human nuclear receptor proteins, such as human nuclear receptor proteins exemplified and referred to throughout this specification as nNR7 and/or nNR7-1. The nuclear receptor proteins encoded by the isolated polynucleotides of the present invention are involved in the regulation of in vivo cell proliferation and/or cell development. Based on amino acid sequence homology, the nuclear receptor that is most related to human nNR7 and/or nNR7-1 with known function is the vitamin D receptor. Northern analysis on the following human tissue samples—heart, brain, placenta, lung, liver, skeletal muscle, kidney, pancreas, adrenal medulla, thyroid, adrenal cortex, testis, thymus, small intestine and stomach—reveals that nNR7 and/or nNR7-1 is expressed mainly in liver at medium to low level and the small intestine. It may be expressed at much higher level in other tissues not examined. This data suggest that nNR7 and/or nNR7-1 plays important roles in carrying out metabolic functions involving D vitamins, since the liver is the major site for generation of hydroxylated D vitamins, which are active forms of vitamin D for the vitamin D receptor. It is also possible that other vitamin D metabolites may be active forms for nNR7 and/or nNR7-1 in the liver.

As noted above, nNR7 and/or nNR7-1 and are expressed in the liver and small intestine. In humans, the cytochrome P-450 monooxygenase 3A4 (CYP3A4) is mainly expressed in the liver and small intestine. The CYP3A4 protein plays an important role in the biotransformation of drugs, including more than 60% of all clinically used drugs, and its expression level is markedly induced by those compounds. Therefore, assays that measure the effects of compounds on CYP3A4 gene expression can predict whether drugs will interact in humans. Because the molecular mechanism underlying this induction is unclear, CYP3A4 gene induction assays have been almost exclusively dependent upon the use of human liver tissue and primary hepatocytes to date. The nNR7 and/or nNR7-1 nuclear receptor disclosed in this application has been disclosed by Lehmann et al. (1998, J. Clin. Invest 102: 1016-1023) subsequent to the priority filing date of this specification. The authors identified a response element located in the CYP3A4 promoter [5′-TGAACT caaagg AGGTCA-3′ (SEQ ID NO:24)] that was shown to bind nNR7 (refered to as hPXR by the authors, but lacking in amino acid 1-32 of nNR7 [SEQ ID NO:2]). The authors suggest that drugs which induce CYP3A4 gene expression activate nNR7 and initiate transcription through CYP3A4 promoter. One of the uses of the DNA molecules and concomitantly expressed proteins of the present invention, including but not limited to nNR7 and nNR7-1, will be useful in assays to identify modulators of CPP3A4 levels in vivo. Therefore, transactivation assays using nNR7 and/or nNR7-1 and theCYP3A4 promoter linked to a reporter gene (such as SEAP—secreted placental alkaline phosphatase) is one approach for identifying modulators of CYP3A4 levels in vivo.

The present invention also relates to isolated nucleic acid fragments which encode mRNA expressing a biologically active novel vertebrate nuclear receptor which belongs to the nuclear receptor superfamily.

A preferred embodiment relates to isolated nucleic acid fragments of SEQ ID NO: 1 and/or SEQ ID NO:17 which encode mRNA expressing a biologically functional derivative of nNR7 and/or nNR7-1, respectively. Any such nucleic acid fragment will encode either a protein or protein fragment comprising at least an intracellular DNA-binding domain and/or ligand binding domain, domains conserved throughout the human nuclear receptor family domain which exist in nNR7 (SEQ ID NO: 2) and/or nNR7-1 (SEQ ID NO:18). Any such polynucleotide includes but is not necessarily limited to nucleotide substitutions, deletions, additions, amino-terminal truncations and carboxy-terminal truncations such that these mutations encode mRNA which express a protein or protein fragment of diagnostic, therapeutic or prophylactic use and would be useful for screening for agonists and/or antagonists of nNR7 and/or nNR7-1.

The isolated nucleic acid molecules of the present invention may include a deoxyribonucleic acid molecule (DNA), such as genomic DNA and complementary DNA (cDNA), which may be single (coding or noncoding strand) or double stranded, as well as synthetic DNA, such as a synthesized, single stranded polynucleotide. The isolated nucleic acid molecule of the present invention may also include a ribonucleic acid molecule (RNA).

The present invention also relates to recombinant vectors and recombinant hosts, both prokaryotic and eukaryotic, which contain the substantially purified nucleic acid molecules disclosed throughout this specification.

A preferred aspect of the present invention is disclosed in FIGS. 1A-C and SEQ ID NO: 1, an isolated human cDNA encoding a novel nuclear trans-acting receptor protein, nNR7.

An especially preferred aspect of the present invention is disclosed in FIGS. 4A-C and SEQ ID NO: 17, an isolated human cDNA encoding a novel nuclear trans-acting receptor protein, nNR7-1.

Another preferred aspect of the present invention relates to a substantially purified form of the novel nuclear trans-acting receptor protein, nNR7, which is disclosed in FIGS. 2A-C and FIG. 3 and as set forth in SEQ ID NO: 2.

Another especially preferred aspect of the present invention relates to a substantially purified form of the novel nuclear trans-acting receptor protein, nNR7-1, which is disclosed in FIGS. 5A-C and FIG. 6 and is set forth as SEQ ID NO: 18. The receptor protein nNR7-1 contains an amino terminal addition compared to nNR7; specifically wherein nNR7-1 contains an intitiating methionine residue as the NH2-terminal amino acid residue.

The present invention also relates to biologically functional derivatives of nNR7 as set forth as SEQ ID NO: 2, including but not limited to nNR7 mutants and biologically active fragments such as amino acid substitutions, deletions, additions, amino terminal truncations and carboxy-terminal truncations, such that these fragments provide for proteins or protein fragments of diagnostic, therapeutic or prophylactic use and would be useful for screening for agonists and/or antagonists of nNR7 function.

The present invention also relates to polyclonal and monoclonal antibodies raised in response to either the human form of nNR7 and/or nNR7-1 disclosed herein, or a biologically functional derivative thereof. It will be especially preferable to raise antibodies against epitopes within the NH₂-terminal domain of nNR7 and/or nNR7-1, which show the least homology to other known proteins belonging to the human nuclear receptor superfamily. To this end, the DNA molecules, RNA molecules, recombinant protein and antibodies of the present invention may be used to screen and measure levels of human nNR7 and/or nNR7-1. The recombinant proteins, DNA molecules, RNA molecules and antibodies lend themselves to the formulation of kits suitable for the detection and typing of human nNR7 and/or nNR7-1.

The present invention also relates to isolated nucleic acid molecules which are fusion constructions expressing fusion proteins useful in assays to identify compounds which modulate wild-type human nNR7 and/or nNR7-1 activity. A preferred aspect of this portion of the invention includes, but is not limited to, glutathione S-transferase GST-nNR7 and/or GST-nNR7-1 fusion constructs. These fusion constructs include, but are not limited to, all or a portion of the ligand-binding domain of nNR7 and/or nNR7-1, respectively, as an in-frame fusion at the carboxy terminus of the GST gene. The disclosure of SEQ ID NOs:1, 2, 17 and 18 allow the artisan of ordinary skill to construct any such nucleic acid molecule encoding a GST-nuclear receptor fusion protein. Soluble recombinant GST-nuclear receptor fusion proteins may be expressed in various expression systems, including Spodoptera frugiperda (Sf21) insect cells (Invitrogen) using a baculovirus expression vector (e.g., Bac-N-Blue DNA from Invitrogen or pAcG2T from Pharmingen). Another preferred aspect of this portion of the invention relates to the use of a receptor/reporter system to identify modulators of nNR7, nNR7-1 and/or CYP3A4 levels in vivo. As an example, and not as a limitation, the portion of SEQ ID NO:1 or SEQ ID NO:17 which encodes the open reading frame of nNR7 and nNR7-1, respectively, may be fused downstream of an active promoter, such as a CMV promoter fragment, so as to overexpress a receptor protein. A second construct includes regulatory regions from the human CYP3A4 gene (such as the human CYP3A4 promoter region or a response element [e.g., SEQ ID NO:24]) fused to a reporter gene, such as SEAP (secreted placental alkaline phosphatase). These constructs may then be utilized in a transactivation assay to identify modulators of CYP3A4 levels in vivo.

It is an object of the present invention to provide an isolated nucleic acid molecule which encodes a novel form of a nuclear receptor protein such as human nNR7 and/or nNR7-1, human nuclear receptor protein fragments of full length proteins such as nNR7 and/or nNR7-1, and mutants which are derivatives of SEQ ID NO: 2 and/or SEQ ID NO:18, respectively. Any such polynucleotide includes but is not necessarily limited to nucleotide substitutions, deletions, additions, amino-terminal truncations and carboxy-terminal truncations such that these mutations encode mRNA which express a protein or protein fragment of diagnostic, therapeutic or prophylactic use and would be useful for screening for agonists and/or antagonists for nNR7 and/or nNR7-1 function.

Another object of this invention is tissue typing using probes or antibodies of this invention. In a particular embodiment, polynucleotide probes are used to identify tissues expressing nNR7 and/or nNR7-1 mRNA. In another embodiment, probes or antibodies can be used to identify a type of tissue based on nNR7 and/or nNR7-1 expression or display of nNR7 and/or nNR7-1 receptors.

It is a further object of the present invention to provide the human nuclear receptor proteins or protein fragments encoded by the nucleic acid molecules referred to in the preceding paragraph.

It is a further object of the present invention to provide recombinant vectors and recombinant host cells which comprise a nucleic acid sequence encoding human nNR7 and/or nNR7-1 or a biological equivalent thereof.

It is an object of the present invention to provide a substantially purified form of nNR7 and/or nNR7-1, as set forth in SEQ ID NO: 2 and SEQ ID NO: 18, respectively.

It is an object of the present invention to provide for biologically functional derivatives of nNR7 and/or nNR7-1, including but not necessarily limited to amino acid substitutions, deletions, additions, amino terminal truncations and carboxy-terminal truncations such that these fragment and/or mutants provide for proteins or protein fragments of diagnostic, therapeutic or prophylactic use.

It is also an object of the present invention to provide for in-frame fusion constructions of nNR7 and/or nNR7-1 and the like, methods of expressing these fusion constructions and biological equivalents disclosed herein, related assays, recombinant cells expressing these constructs and agonistic and/or antagonistic compounds identified through the use DNA molecules encoding human nuclear receptor proteins such as nNR7 and/or nNR7-1.

As used herein, “DBD” refers to DNA binding domain.

As used herein, “LBD” refers to ligand binding domain.

As used herein, the term “mammalian host” refers to any mammal, including a human being.

### BRIEF DESCRIPTION OF THE DRAWINGS

FIGS. 1A-C shows the nucleotide sequence which comprises the open reading frame encoding the human nuclear receptor protein, nNR7 (SEQ ID NO: 1).

FIGS. 2A-C shows the nucleotide sequence of the isolated cDNA molecule (SEQ ID NO: 1) which encodes nNR7, and the amino acid sequence of (SEQ ID NO: 2) nNR7. The region in bold and underline is the DNA binding domain.

FIG. 3 shows the amino acid sequence of nNR7 (SEQ ID NO: 3). The region in bold and underline is the DNA binding domain.

FIGS. 4A-C shows the nucleotide sequence which comprises the open reading frame encoding the human nuclear receptor protein, nNR7 (SEQ ID NO: 1). The region of nucleotides which are underlined represent the extended 5′-end of the nNR7 cDNA clone (as shown in SEQ ID NO:1 and FIGS. 1A-C).

FIGS. 5A-C shows the nucleotide sequence of the isolated cDNA molecule (SEQ ID NO: 17) which encodes nNR7-1, and the amino acid sequence of (SEQ ID NO:18) nNR7-1. The region of nucleotides which are underlined represent the extended 5′-end of the nNR7 cDNA clone (as shown in SEQ ID NO:1 and FIGS. 1A-C). The amino acid region underlined represents the DNA binding domain.

FIG. 6 shows the amino acid sequence of nNR7-1 (SEQ ID NO: 18). The region in bold is the DNA binding domain. The underline indicates the extended N-terminus of nNR7-1 as compared to nNR7.

### DETAILED DESCRIPTION OF THE INVENTION

The present invention relates to isolated nucleic acid and protein forms which represent nuclear receptors, preferably but not necessarily limited to human receptors. These expressed proteins are novel nuclear receptors and which are useful in the identification of downstream target genes and ligands regulating their activity. The nuclear receptor proteins encoded by the isolated polynucleotides of the present invention are involved in the regulation of in vivo cell proliferation and/or cell development. The nuclear receptor superfamily is composed of a group of structurally related receptors which are regulated by chemically distinct ligands. The common structure for a nuclear receptor is a highly conserved DNA binding domain (DBD) located in the center of the peptide and the ligand-binding domain (LBD) at the COOH-terminus. Eight out of the nine non-variant cysteines form two type II zinc fingers which distinguish nuclear receptors from other DNA-binding proteins. The DBDs share at least 50% to 60% amino acid sequence identity even among the most distant members in vertebrates. The superfamily has been expanded within the past decade to contain approximately 25 subfamilies. A first exemplified cDNA encoding human nNR was identified via low stringency screening on a mixed cDNA library. The probes used in the screening were DNA fragments corresponding to AR, ERb, GR and VDR DNA binding domain regions and 250,000 primary clones from the mixed library were screened. After two rounds of screening, in vivo excision was carried out on the purified lZAPII phages which showed positive signals at low stringency condition. Plasmid DNA samples were submitted to sequence directly using M13 forward and reverse primers. A blast search of the sequence information was obtained using computer program PhredPhrap. A single cDNA clone, referred to as gm6, is shown herein to encode a novel human nuclear receptor, nNR7. DNA sequencing was continued from both 5′ and 3′ ends on nNR7. An intron was identified when sequencing with pgm6y2 (5′-CTTCAATGTCATGACATG-3′; SEQ ID NO: 3). A cDNA fragment flanking the intron region was retrieved via PCR using primer pair of NR7F (5′-CCAAATCTGCCGTGTATGTG-3′; SEQ ID NO: 4) and pgm6xC (5′-GTCAGTGCACTCTCCACGT-3′; SEQ ID NO: 5) followed by double nested primers of pgm6y2 and pgm6xD (5′-TGCAGCTGGTCCACCACGCG-3′; SEQ ID NO: 6). A prominent DNA fragment of ˜1.5 kb was amplified form testis and brain cDNAs. The fragment was purified and subcloned into pCRII vector (Invitrogen, San Diego, Calif.). Automated sequencing was performed on several of the clones. The complete sequencing of gm6 clone revealed a 3.1 kb cDNA which codes an authentic novel nuclear receptor. The open reading frame for the expressed protein does not contain an initiating methionine.

Primer pair pgm6xAR (5′-GGGTATGCTCTGTGACAAG-3′; SEQ ID NO: 7) and pgm6x (5′-AGGCAGGCACTTTCATACC-3′; SEQ ID NO: 8) in 3′ non-coding region were used to scan the 83 clones of the Stanford radiation hybrid panel (Cox et al., 1990, Science, 250:245:250). The PCR results were scored and submitted to the Stanford Genome Center for linkage analysis. The result indicated that nNR7 was located on chromosome 3. Northern analysis on Clontech blots showed that nNR7 was mainly expressed in human liver at medium to low level. The nNR7 polypeptide has a 42% sequence identity at the amino acid level with human VDR (Baker, et al., 1988, Proc. Natl. Acad. Sci. (U.S.A) 85 (10): 3294-3298) in the overlapping regions. Additionally, the nNR7 polypeptide has a 52% sequence identity at the amino acid level with human hONR (Baes et al., 1994, Mol. Cell. Biol. 14(3):1544-1552) in the overlapping regions.

The present invention also relates to isolated nucleic acid fragments of nNR7 (SEQ ID NO: 1) which encode mRNA expressing a biologically active novel human nuclear receptor. Any such nucleic acid fragment will encode either a protein or protein fragment comprising at least an intracellular DNA-binding domain and/or ligand binding domain, domains conserved throughout the human nuclear receptor family domain which exist in nNR7 (SEQ ID NO: 2). Any such polynucleotide includes but is not necessarily limited to nucleotide substitutions, deletions, additions, amino-terminal truncations and carboxy-terminal truncations such that these mutations encode mRNA which express a protein or protein fragment of diagnostic, therapeutic or prophylactic use and would be useful for screening for agonists and/or antagonists for nNR7 function.

The isolated nucleic acid molecule of the present invention may include a deoxyribonucleic acid molecule (DNA), such as genomic DNA and complementary DNA (cDNA), which may be single (coding or noncoding strand) or double stranded, as well as synthetic DNA, such as a synthesized, single stranded polynucleotide. The isolated nucleic acid molecule of the present invention may also include a ribonucleic acid molecule (RNA).

The present invention also relates to recombinant vectors and recombinant hosts, both prokaryotic and eukaryotic, which contain the substantially purified nucleic acid molecules disclosed throughout this specification.

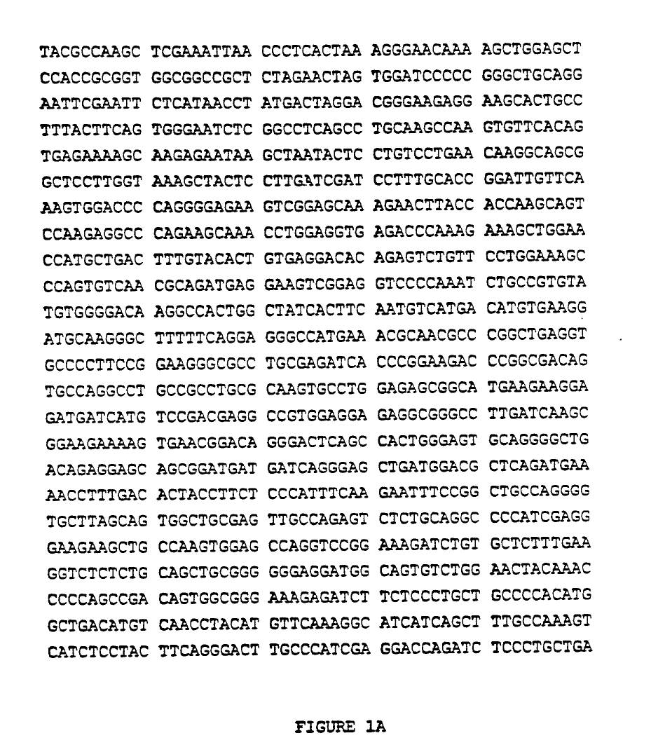

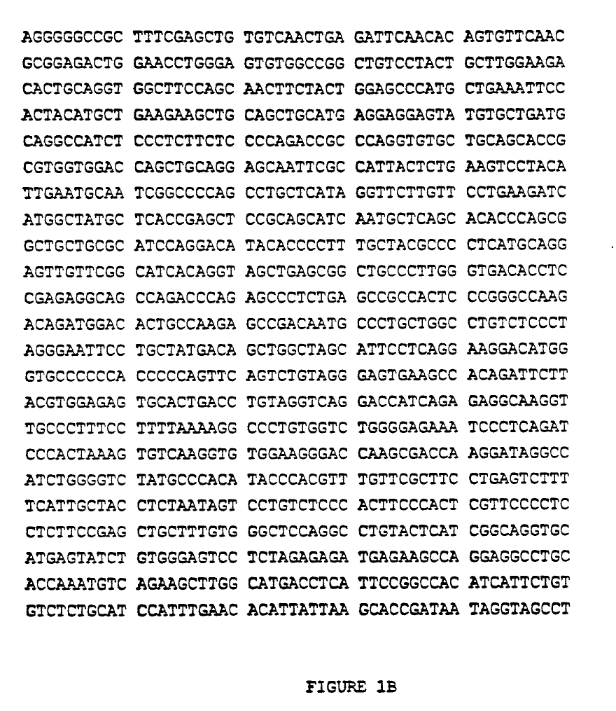

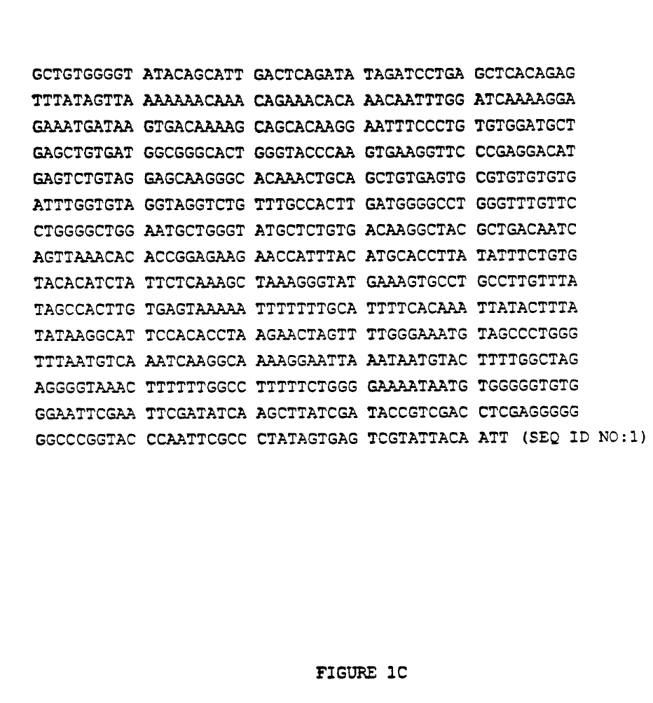

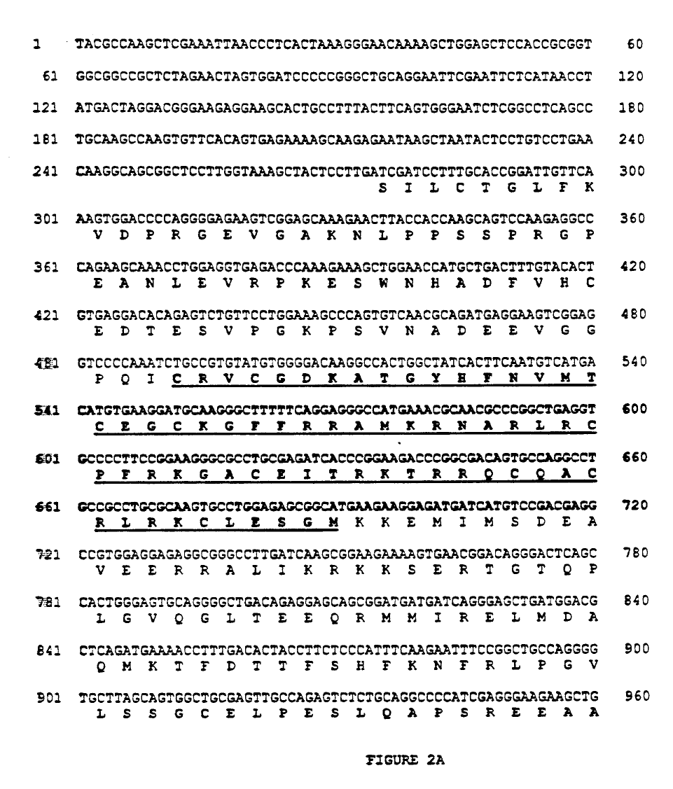

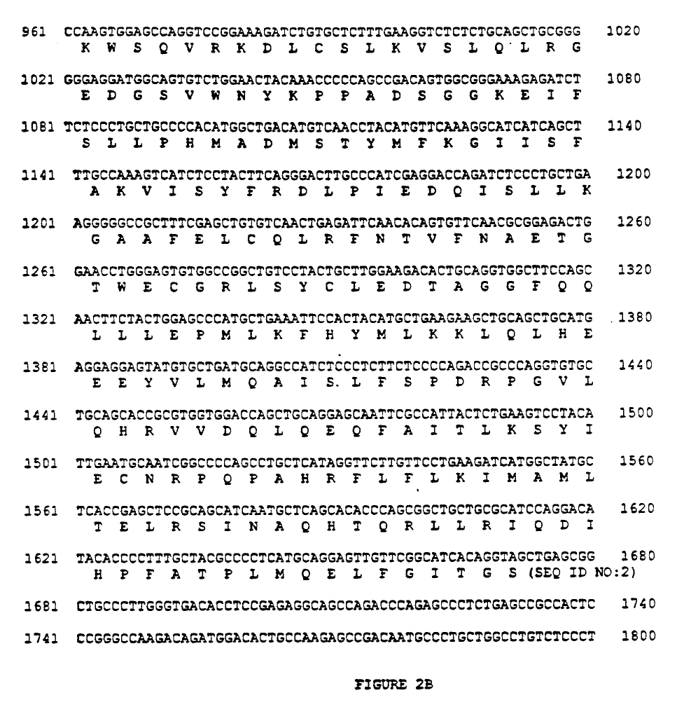

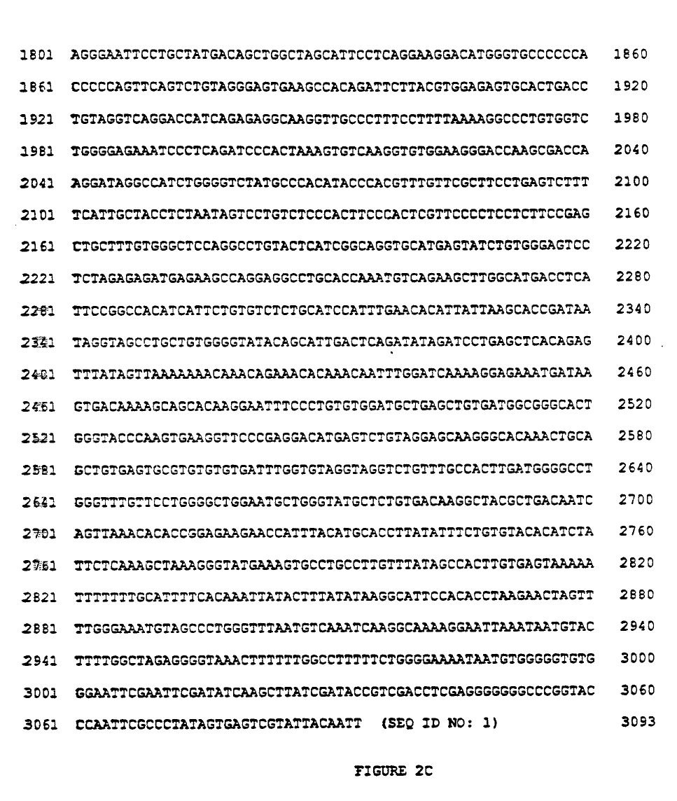

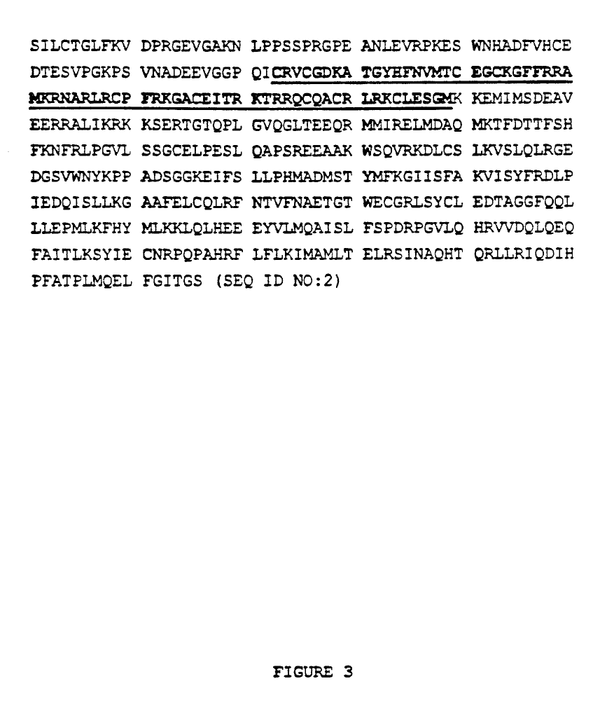

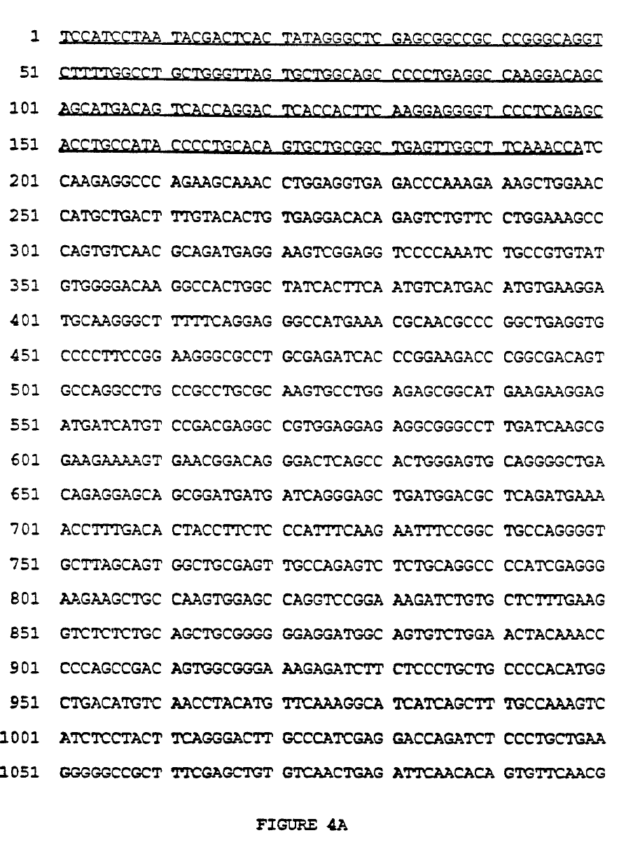

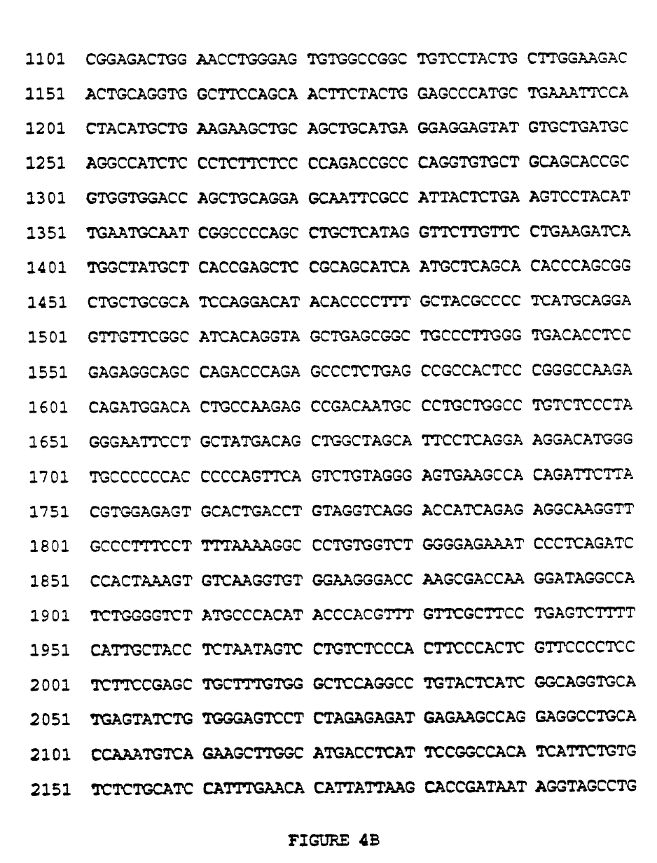

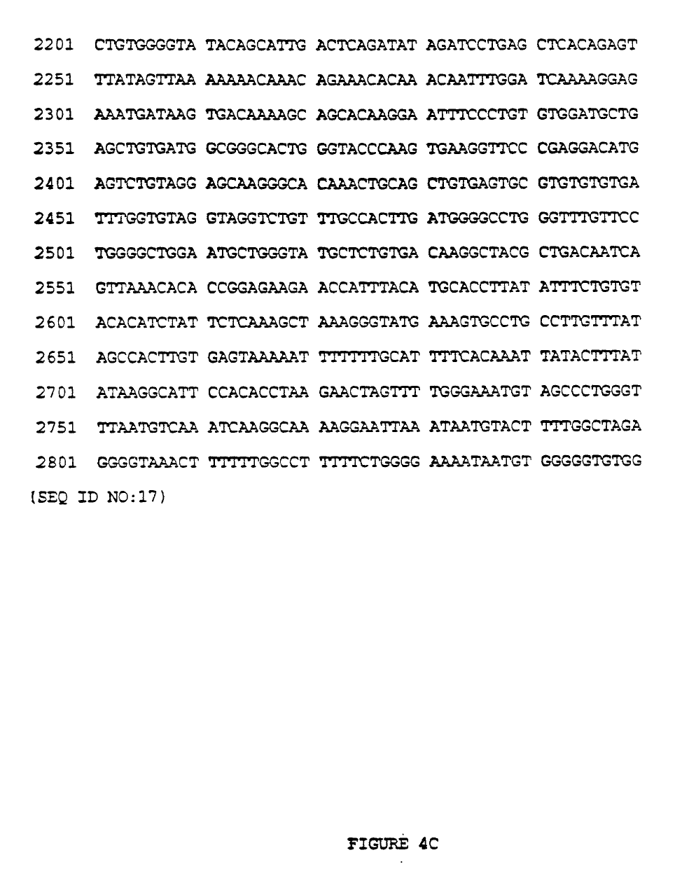

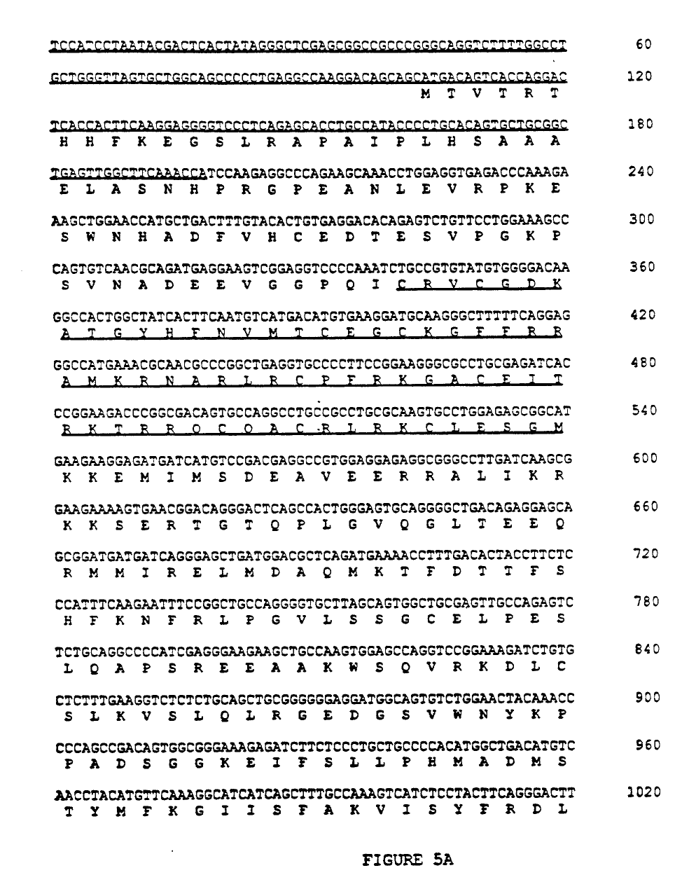

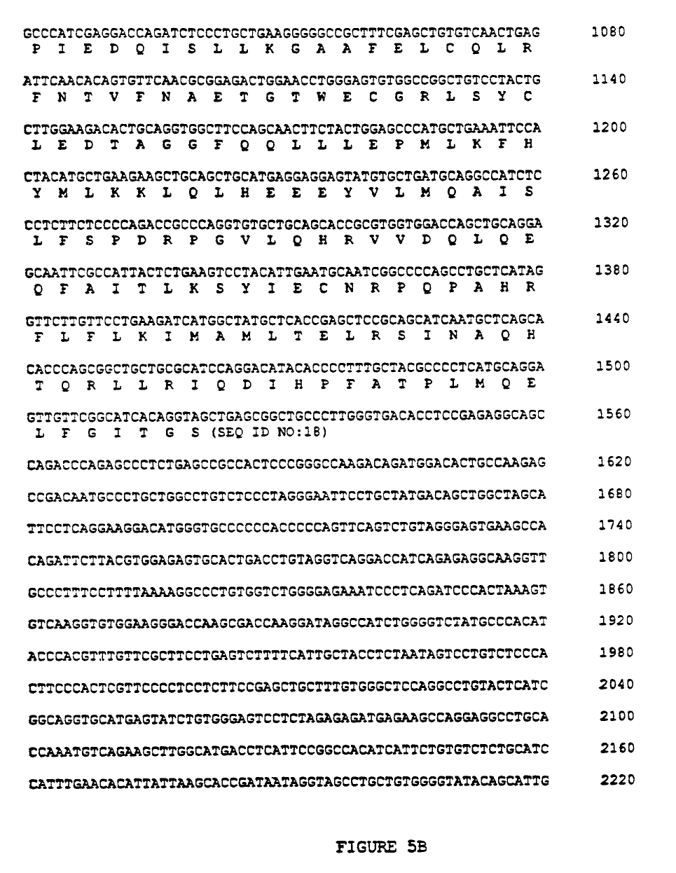

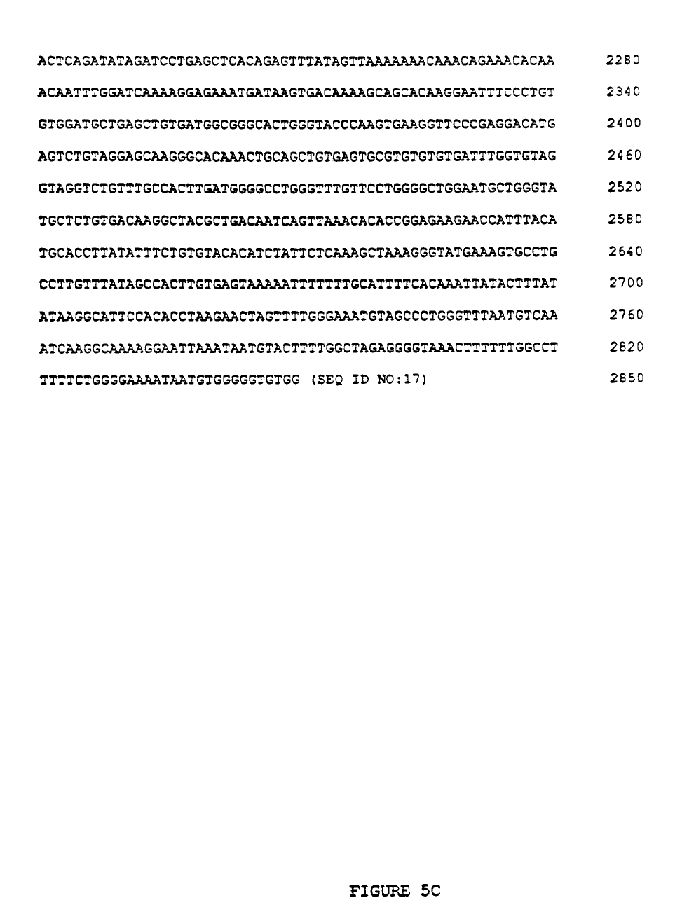

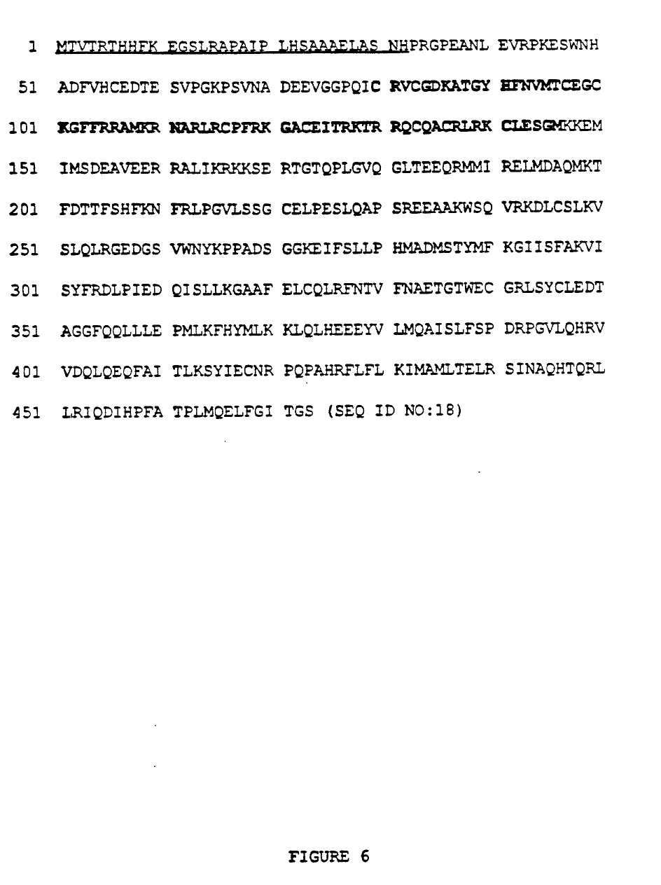

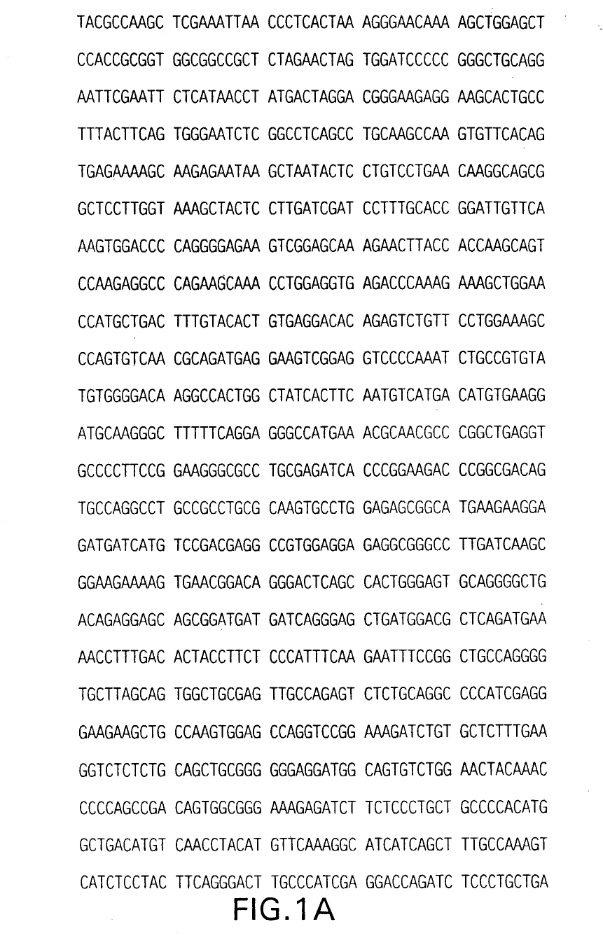

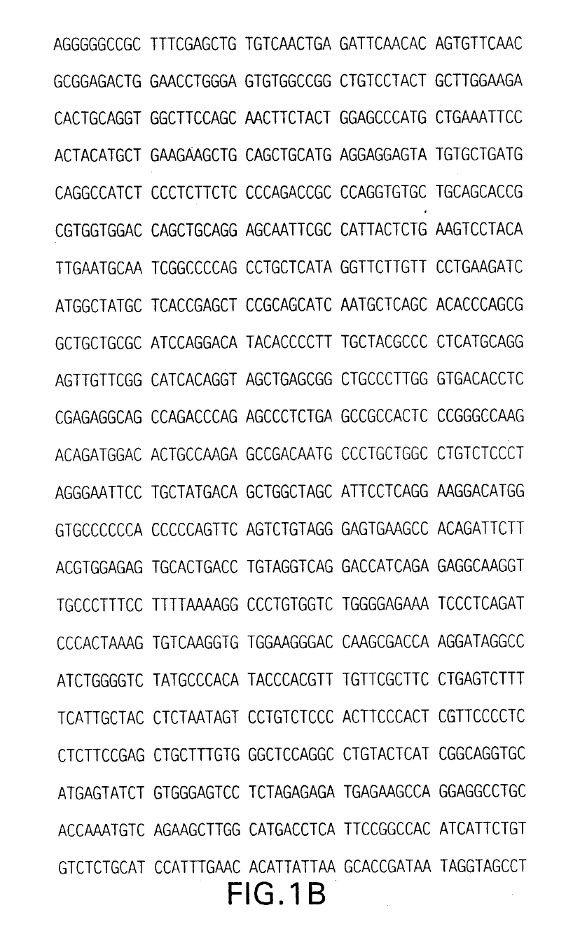

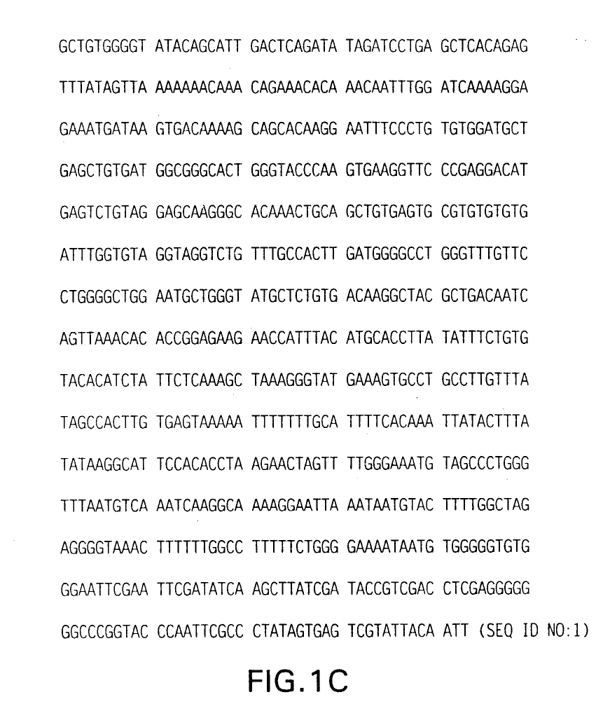

A preferred aspect of the present invention is disclosed in FIGS. 1A-C and SEQ ID NO: 1, a human cDNA encoding a novel nuclear trans-acting receptor protein, nNR7, disclosed as follows:

(SEQ ID NO:1)
TACGCCAAGC TCGAAATTAA CCCTCACTAA AGGGAACAAA
AGCTGGAGCT
CCACCGCGGT GGCGGCCGCT CTAGAACTAG TGGATCCCCC
GGGCTGCAGG
AATTCGAATT CTCATAACCT ATGACTAGGA CGGGAAGAGG
AAGCACTGCC
TTTACTTCAG TGGGAATCTC GGCCTCAGCC TGCAAGCCAA
GTGTTCACAG
TGAGAAAAGC AAGAGAATAA GCTAATACTC CTGTCCTGAA
CAAGGCAGCG
GCTCCTTGGT AAAGCTACTC CTTGATCGAT CCTTTGCACC
GGATTGTTCA
AAGTGGACCC CAGGGGAGAA GTCGGAGCAA AGAACTTACC
ACCAAGCAGT
CCAAGAGGCC CAGAAGCAAA CCTGGAGGTG AGACCCAAAG
AAAGCTGGAA
CCATGCTGAC TTTGTACACT GTGAGGACAC AGAGTCTGTT
CCTGGAAAGC
CCAGTGTCAA CGCAGATGAG GAAGTCGGAG GTCCCCAAAT
CTGCCGTGTA
TGTGGGGACA AGGCCACTGG CTATCACTTC AATGTCATGA
CATGTGAAGG
ATGCAAGGGC TTTTTCAGGA GGGCCATGAA ACGCAACGCC
CGGCTGAGGT
GCCCCTTCCG GAAGGGCGCC TGCGAGATCA CCCGGAAGAC
CCGGCGACAG
TGCCAGGCCT GCCGCCTGCG CAAGTGCCTG GAGAGCGGCA
TGAAGAAGGA
GATGATCATG TCCGACGAGG CCGTGGAGGA GAGGCGGGCC
TTGATCAAGC
GGAAGAAAAG TGAACGGACA GGGACTCAGC CACTGGGAGT
GCAGGGGCTG
ACAGAGGAGC AGCGGATGAT GATCAGGGAG CTGATGGACG
CTCAGATGAA
AACCTTTGAC ACTACCTTCT CCCATTTCAA GAATTTCCGG
CTGCCAGGGG
TGCTTAGCAG TGGCTGCGAG TTGCCAGAGT CTCTGCAGGC
CCCATCGAGG
GAAGAAGCTG CCAAGTGGAG CCAGGTCCGG AAAGATCTGT
GCTCTTTGAA
GGTCTCTCTG CAGCTGCGGG GGGAGGATGG CAGTGTCTGG
AACTACAAAC
CCCCAGCCGA CAGTGGCGGG AAAGAGATCT TCTCCCTGCT
GCCCCACATG
GCTGACATGT CAACCTACAT GTTCAAAGGC ATCATCAGCT
TTGCCAAAGT
CATCTCCTAC TTCAGGGACT TGCCCATCGA GGACCAGATC
TCCCTGCTGA
AGGGGGCCGC TTTCGAGCTG TGTCAACTGA GATTCAACAC
AGTGTTCAAC
GCGGAGACTG GAACCTGGGA GTGTGGCCGG CTGTCCTACT
GCTTGGAAGA
CACTGCAGGT GGCTTCCAGC AACTTCTACT GGAGCCCATG
CTGAAATTCC
ACTACATGCT GAAGAAGCTG CAGCTGCATG AGGAGGAGTA
TGTGCTGATG
CAGGCCATCT CCCTCTTCTC CCCAGACCGC CCAGGTGTGC
TGCAGCACCG
CGTGGTGGAC CAGCTGCAGG AGCAATTCGC CATTACTCTG
AAGTCCTACA
TTGAATGCAA TCGGCCCCAG CCTGCTCATA GGTTCTTGTT
CCTGAAGATC
ATGGCTATGC TCACCGAGCT CCGCAGCATC AATGCTCAGC
ACACCCAGCG
GCTGCTGCGC ATCCAGGACA TACACCCCTT TGCTACGCCC
CTCATGCAGG
AGTTGTTCGG CATCACAGGT AGCTGAGCGG CTGCCCTTGG
GTGACACCTC
CGAGAGGCAG CCAGACCCAG AGCCCTCTGA GCCGCCACTC
CCGGGCCAAG
ACAGATGGAC ACTGCCAAGA GCCGACAATG CCCTGCTGGC
CTGTCTCCCT
AGGGAATTCC TGCTATGACA GCTGGCTAGC ATTCCTCAGG
AAGGACATGG
GTGCCCCCCA CCCCCAGTTC AGTCTGTAGG GAGTGAAGCC
ACAGATTCTT
ACGTGGAGAG TGCACTGACC TGTAGGTCAG GACCATCAGA
GAGGCAAGGT
TGCCCTTTCC TTTTAAAAGG CCCTGTGGTC TGGGGAGAAA
TCCCTCAGAT
CCCACTAAAG TGTCAAGGTG TGGAAGGGAC CAAGCGACCA
AGGATAGGCC
ATCTGGGGTC TATGCCCACA TACCCACGTT TGTTCGCTTC
CTGAGTCTTT
TCATTGCTAC CTCTAATAGT CCTGTCTCCC ACTTCCCACT
CGTTCCCCTC
CTCTTCCGAG CTGCTTTGTG GGCTCCAGGC CTGTACTCAT
CGGCAGGTGC
ATGAGTATCT GTGGGAGTCC TCTAGAGAGA TGAGAAGCCA
GGAGGCCTGC
ACCAAATGTC AGAAGCTTGG CATGACCTCA TTCCGGCCAC
ATCATTCTGT
GTCTCTGCAT CCATTTGAAC ACATTATTAA GCACCGATAA
TAGGTAGCCT
GCTGTGGGGT ATACAGCATT GACTCAGATA TAGATCCTGA
GCTCACAGAG
TTTATAGTTA AAAAAACAAA CAGAAACACA AACAATTTGG
ATCAAAAGGA
GAAATGATAA GTGACAAAAG CAGCACAAGG AATTTCCCTG
TGTGGATGCT
GAGCTGTGAT GGCGGGCACT GGGTACCCAA GTGAAGGTTC
CCGAGGACAT
GAGTCTGTAG GAGCAAGGGC ACAAACTGCA GCTGTGAGTG
CGTGTGTGTG
ATTTGGTGTA GGTAGGTCTG TTTGCCACTT GATGGGGCCT
GGGTTTGTTC
CTGGGGCTGG AATGCTGGGT ATGCTCTGTG ACAAGGCTAC
GCTGACAATC
AGTTAAACAC ACCGGAGAAG AACCATTTAC ATGCACCTTA
TATTTCTGTG
TACACATCTA TTCTCAAAGC TAAAGGGTAT GAAAGTGCCT
GCCTTGTTTA
TAGCCACTTG TGAGTAAAAA TTTTTTTGCA TTTTCACAAA
TTATACTTTA
TATAAGGCAT TCCACACCTA AGAACTAGTT TTGGGAAATG
TAGCCCTGGG
TTTAATGTCA AATCAAGGCA AAAGGAATTA AATAATGTAC
TTTTGGCTAG
AGGGGTAAAC TTTTTTGGCC TTTTTCTGGG GAAAATAATG
TGGGGGTGTG
GGAATTCGAA TTCGATATCA AGCTTATCGA TACCGTCGAC
CTCGAGGGGG
GGCCCGGTAC CCAATTCGCC CTATAGTGAG TCGTATTACA
ATT.

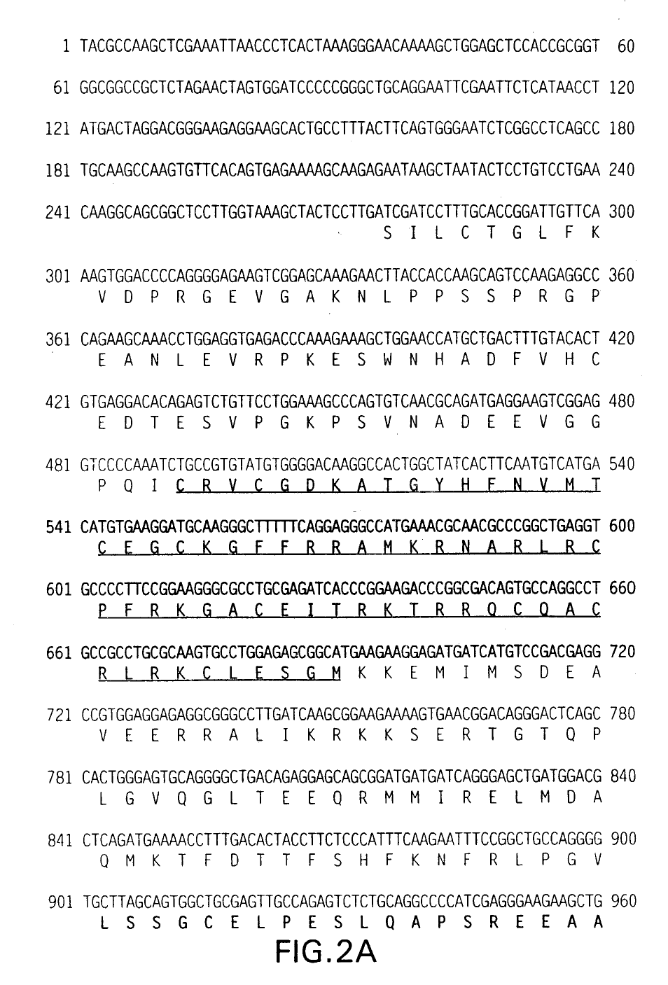

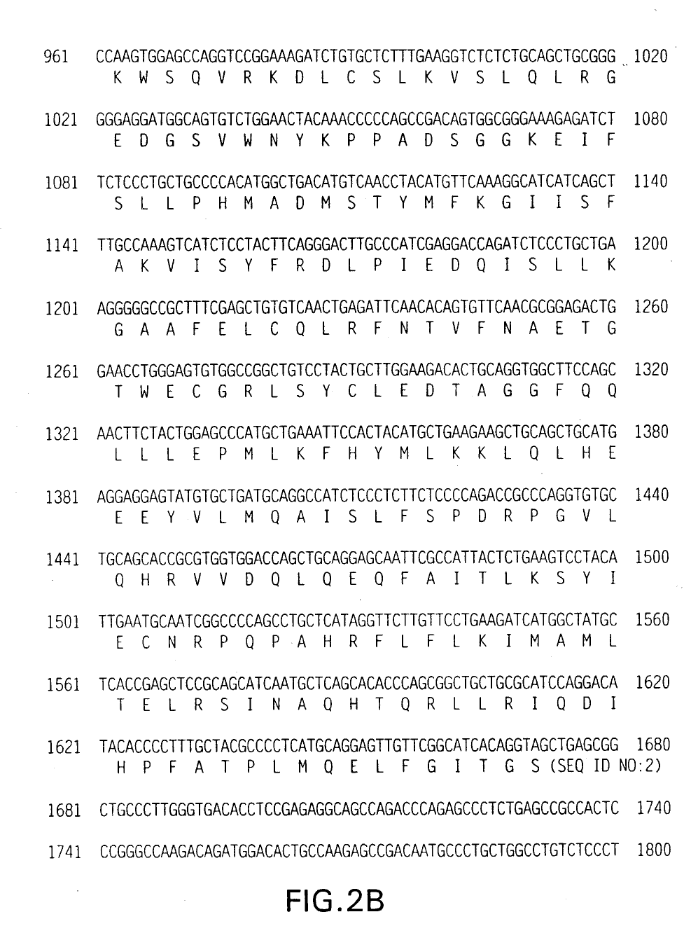

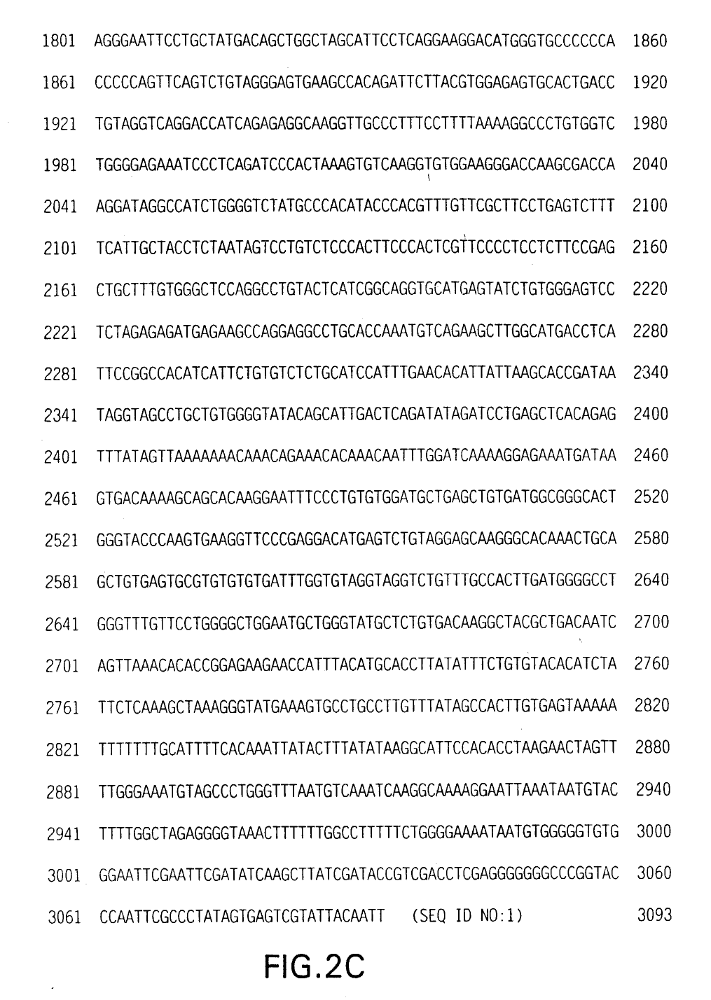

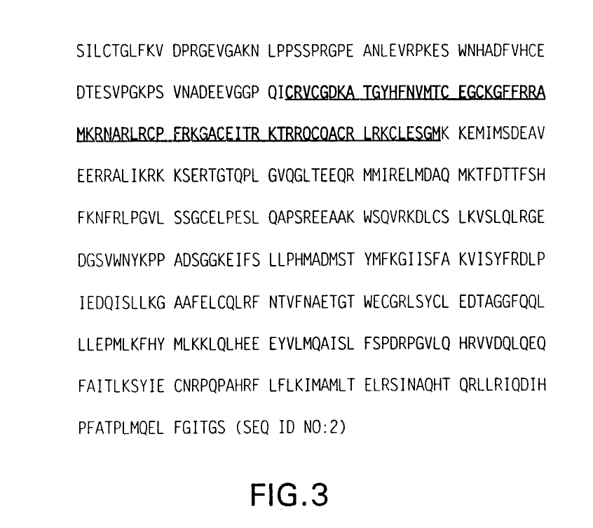

The above-exemplified isolated DNA molecule, shown in FIGS. 1A-C and set forth as SEQ ID NO:1, contains an open reading frame from nucleotide 276 to nucleotide 1673, with a “TGA” termination codon from nucleotides 1674-1676. This open reading frame encodes a human nNR7 receptor, a 466 amino acid protein shown in FIGS. 2A-C and FIG. 3 and as set forth in SEQ ID NO:2.

Therefore, the present invention also relates to a substantially purified form of the novel nuclear trans-acting receptor protein, nNR7, shown in FIGS. 2A-C and FIG. 3 and as set forth in SEQ ID NO: 2, disclosed as follows:

(SEQ ID NO:2)
SILCTGLFKV DPRGEVGAKN LPPSSPRGPE ANLEVRPKES
WNHADFVHCE
DTESVPGKPS VNADEEVGGP QICRVCGDKA TGYHFNVMTC
EGCKGFFRRA
MKRNARLRCP FRKGACEITR KTRRQCQACR LRKCLESGMK
KEMIMSDEAV
EERRALIKRK KSERTGTQPL GVQGLTEEQR MMIRELMDAQ
MKTFDTTFSH
FKNFRLPGVL SSGCELPESL QAPSREEAAK WSQVRKDLCS
LKVSLQLRGE
DGSVWNYKPP ADSGGKEIFS LLPHMADMST YMFKGIISFA
KVISYFRDLP
IEDQISLLKG AAFELCQLRF NTVFNAETGT WECGRLSYCL
EDTAGGFQQL
LLEPMLKFHY MLKKLQLHEE EYVLMQAISL FSPDRPGVLQ
HRVVDQLQEQ
FAITLKSYIE CNRPQPAHRF LFLKIMAMLT ELRSINAQHT
QRLLRIQDIH

PFATPLMQEL FGITGS.

The present invention also relates to biologically functional derivatives and/or mutants of nNR7 as set forth as SEQ ID NO: 2, including but not necessarily limited to amino acid substitutions, deletions, additions, amino terminal truncations and carboxy-terminal truncations such that these mutations provide for proteins or protein fragments of diagnostic, therapeutic or prophylactic use and would be useful for screening for agonists and/or antagonists of nNR7 function.

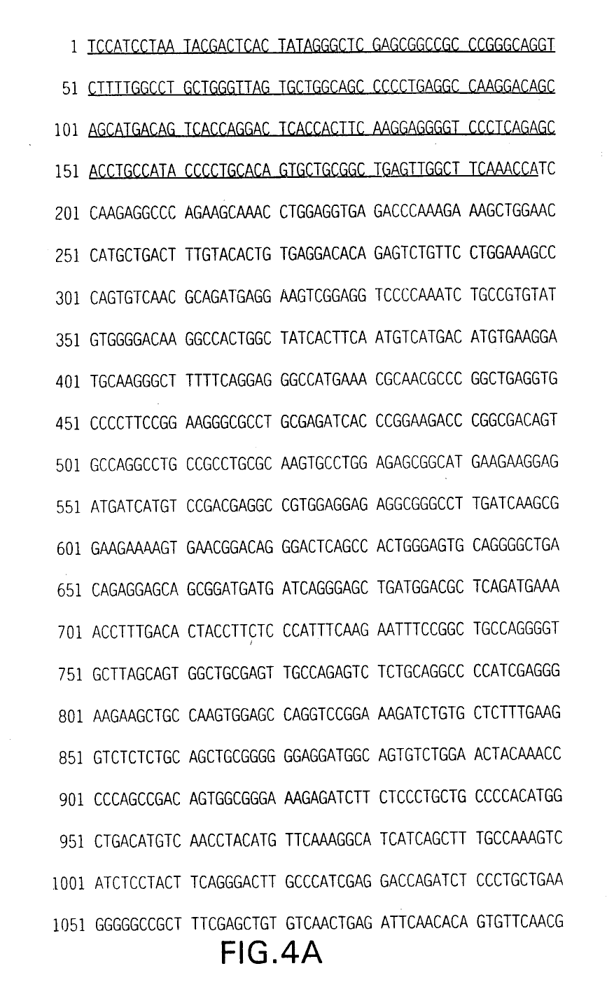

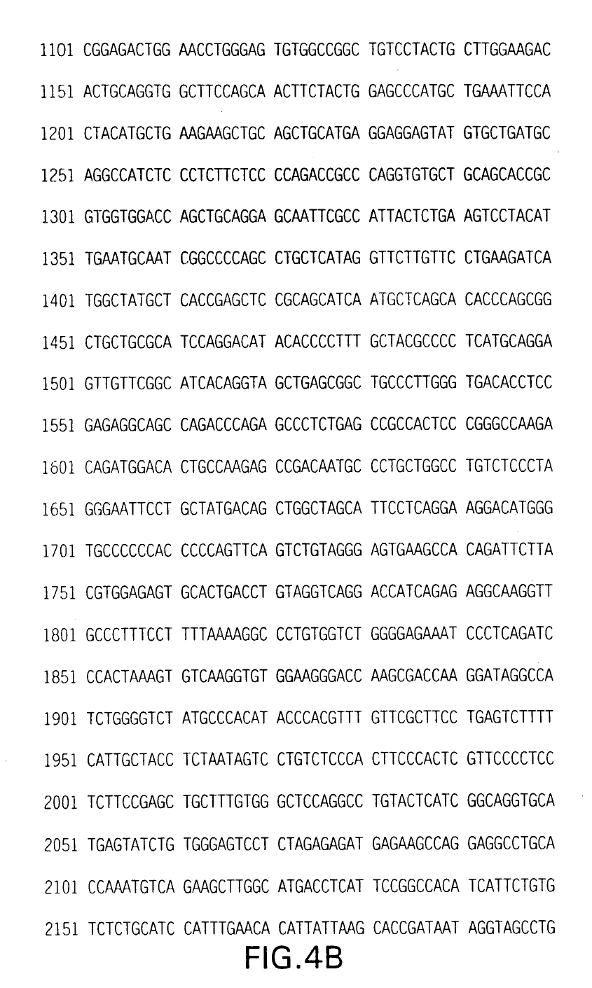

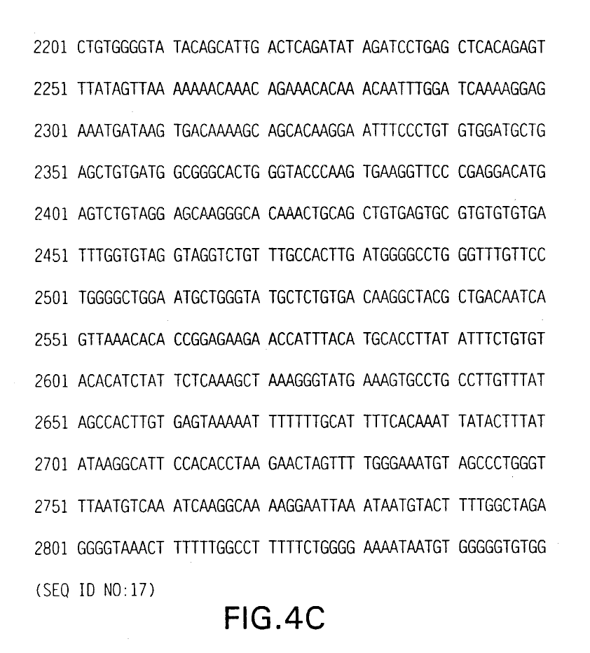

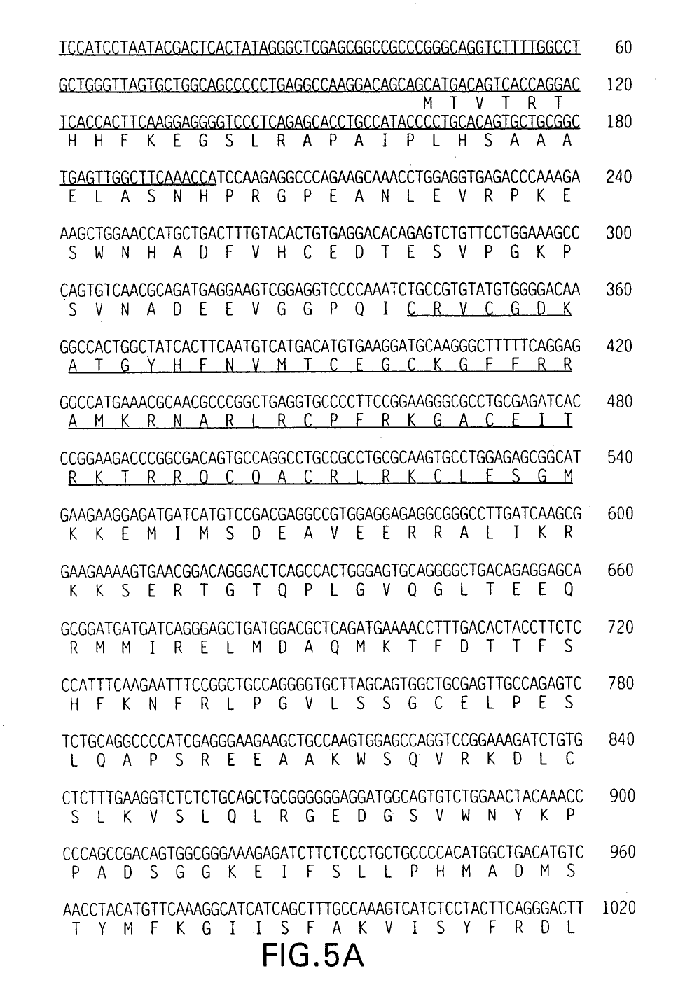

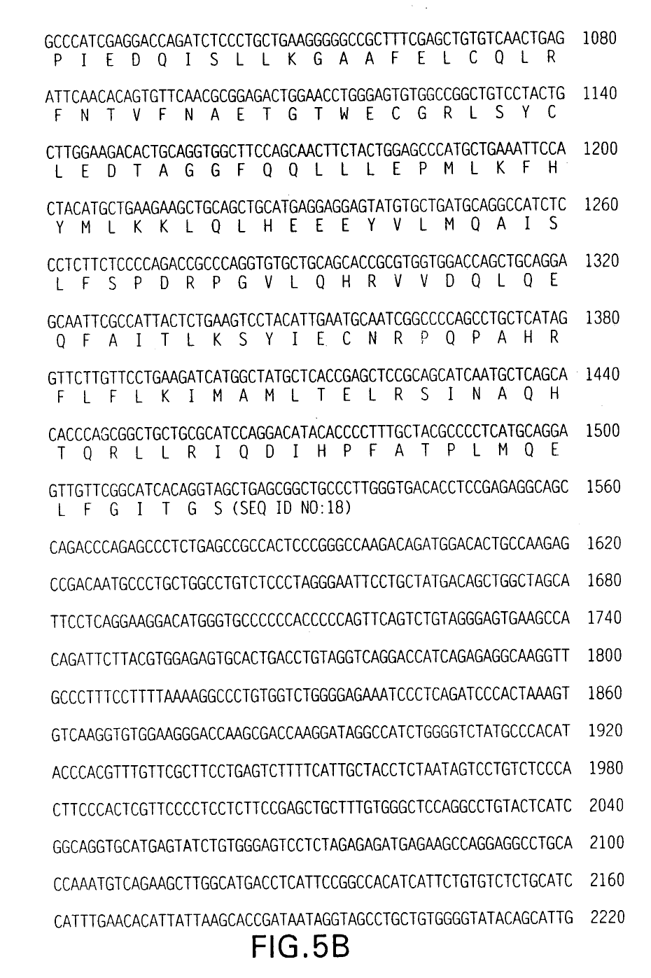

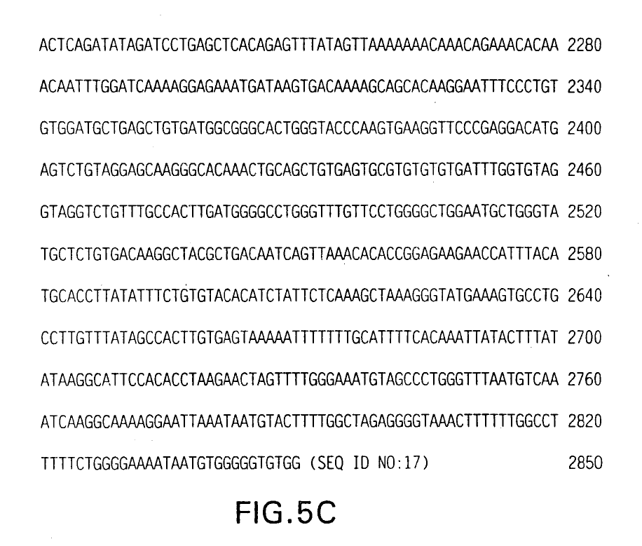

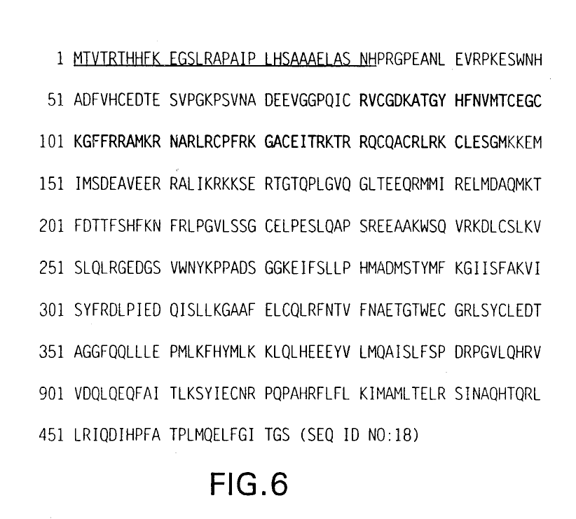

Another exemplification of the present invention relates to an isolated DNA molecule which comprises the nucleotide sequence as disclosed in FIGS. 4A-C and as set forth in SEQ ID NO:17. The initial exemplified cDNA molecule disclosed in FIGS. 1A-C and set forth as SEQ ID NO:1 does not contain a starting Met. Therefore, extension of the nNR7 5′-end sequence was continued. Three clones from 5′-end RACE (rapid amplification of cDNA end) showed divergence from the original cDNA as set forth in SEQ ID NO:1. One of these clones was shown to contain an intitating Met with an inframe stop codon in front. This DNA molecule is set forth herein in FIGS. 4A-C and is set forth as SEQ ID NO:17. This DNA molecule encodes a complete reading frame for an nNR7 type nuclear receptor and is refered herein as nNR7-1, and is set forth in FIGS. 5A-C and FIG. 6, as well as SEQ ID NO:18.

This portion of the present invention also relates to sent invention relates to isolated nucleic acid fragments of SEQ ID NO: 17 which encode mRNA expressing a biologically active novel human nuclear receptor. Any such nucleic acid fragment will encode either a protein or protein fragment comprising at least an intracellular DNA-binding domain and/or ligand binding domain, domains conserved throughout the human nuclear receptor family domain which exist in nNR7-1 (SEQ ID NO: 18). Any such polynucleotide includes but is not necessarily limited to nucleotide substitutions, deletions, additions, amino-terminal truncations and carboxy-terminal truncations such that these mutations encode mRNA which express a protein or protein fragment of diagnostic, therapeutic or prophylactic use and would be useful for screening for agonists and/or antagonists for nNR7-1 function.

The isolated nucleic acid molecule of this portion of the invention may include a deoxyribonucleic acid molecule (DNA), such as genomic DNA and complementary DNA (cDNA), which may be single (coding or noncoding strand) or double stranded, as well as synthetic DNA, such as a synthesized, single stranded polynucleotide. The isolated nucleic acid molecule of the present invention may also include a ribonucleic acid molecule (RNA).

The present invention also relates to recombinant vectors and recombinant hosts, both prokaryotic and eukaryotic, which contain the substantially purified nucleic acid molecules disclosed throughout this specification.

An especially preferred aspect of the present invention is disclosed in FIGS. 4A-C and SEQ ID NO: 17, a human cDNA encoding a novel nuclear trans-acting receptor protein, nNR7-1, disclosed as follows:

(SEQ ID NO:17)
TCCATCCTAA TACGACTCAC TATAGGGCTC GAGCGGCCGC
CCGGGCAGGT
CTTTTGGCCT GCTGGGTTAG TGCTGGCAGC CCCCTGAGGC
CAAGGACAGC
AGCATGACAG TCACCAGGAC TCACCACTTC AAGGAGGGGT
CCCTCAGAGC
ACCTGCCATA CCCCTGCACA GTGCTGCGGC TGAGTTGGCT
TCAAACCATC
CAAGAGGCCC AGAAGCAAAC CTGGAGGTGA GACCCAAAGA
AAGCTGGAAC
CATGCTGACT TTGTACACTG TGAGGACACA GAGTCTGTTC
CTGGAAAGCC
CAGTGTCAAC GCAGATGAGG AAGTCGGAGG TCCCCAAATC
TGCCGTGTAT
GTGGGGACAA GGCCACTGGC TATCACTTCA ATGTCATGAC
ATGTGAAGGA
TGCAAGGGCT TTTTCAGGAG GGCCATGAAA CGCAACGCCC
GGCTGAGGTG
CCCCTTCCGG AAGGGCGCCT GCGAGATCAC CCGGAAGACC
CGGCGACAGT
GCCAGGCCTG CCGCCTGCGC AAGTGCCTGG AGAGCGGCAT
GAAGAAGGAG
ATGATCATGT CCGACGAGGC CGTGGAGGAG AGGCGGGCCT
TGATCAAGCG
GAAGAAAAGT GAACGGACAG GGACTCAGCC ACTGGGAGTG
CAGGGGCTGA
CAGAGGAGCA GCGGATGATG ATCAGGGAGC TGATGGACGC
TCAGATGAAA
ACCTTTGACA CTACCTTCTC CCATTTCAAG AATTTCCGGC
TGCCAGGGGT
GCTTAGCAGT GGCTGCGAGT TGCCAGAGTC TCTGCAGGCC
CCATCGAGGG
AAGAAGCTGC CAAGTGGAGC CAGGTCCGGA AAGATCTGTG
CTCTTTGAAG
GTCTCTCTGC AGCTGCGGGG GGAGGATGGC AGTGTCTGGA
ACTACAAACC
CCCAGCCGAC AGTGGCGGGA AAGAGATCTT CTCCCTGCTG
CCCCACATGG
CTGACATGTC AACCTACATG TTCAAAGGCA TCATCAGCTT
TGCCAAAGTC
ATCTCCTACT TCAGGGACTT GCCCATCGAG GACCAGATCT
CCCTGCTGAA
GGGGGCCGCT TTCGAGCTGT GTCAACTGAG ATTCAACACA
GTGTTCAACG
CGGAGACTGG AACCTGGGAG TGTGGCCGGC TGTCCTACTG
CTTGGAAGAC
ACTGCAGGTG GCTTCCAGCA ACTTCTACTG GAGCCCATGC
TGAAATTCCA
CTACATGCTG AAGAAGCTGC AGCTGCATGA GGAGGAGTAT
GTGCTGATGC
AGGCCATCTC CCTCTTCTCC CCAGACCGCC CAGGTGTGCT
GCAGCACCGC
GTGGTGGACC AGCTGCAGGA GCAATTCGCC ATTACTCTGA
AGTCCTACAT
TGAATGCAAT CGGCCCCAGC CTGCTCATAG GTTCTTGTTC
CTGAAGATCA
TGGCTATGCT CACCGAGCTC CGCAGCATCA ATGCTCAGCA
CACCCAGCGG
CTGCTGCGCA TCCAGGACAT ACACCCCTTT GCTACGCCCC
TCATGCAGGA
GTTGTTCGGC ATCACAGGTA GCTGAGCGGC TGCCCTTGGG
TGACACCTCC
GAGAGGCAGC CAGACCCAGA GCCCTCTGAG CCGCCACTCC
CGGGCCAAGA
CAGATGGACA CTGCCAAGAG CCGACAATGC CCTGCTGGCC
TGTCTCCCTA
GGGAATTCCT GCTATGACAG CTGGCTAGCA TTCCTCAGGA
AGGACATGGG
TGCCCCCCAC CCCCAGTTCA GTCTGTAGGG AGTGAAGCCA
CAGATTCTTA
CGTGGAGAGT GCACTGACCT GTAGGTCAGG ACCATCAGAG
AGGCAAGGTT
GCCCTTTCCT TTTAAAAGGC CCTGTGGTCT GGGGAGAAAT
CCCTCAGATC
CCACTAAAGT GTCAAGGTGT GGAAGGGACC AAGCGACCAA
GGATAGGCCA
TCTGGGGTCT ATGCCCACAT ACCCACGTTT GTTCGCTTCC
TGAGTCTTTT
CATTGCTACC TCTAATAGTC CTGTCTCCCA CTTCCCACTC
GTTCCCCTCC
TCTTCCGAGC TGCTTTGTGG GCTCCAGGCC TGTACTCATC
GGCAGGTGCA
TGAGTATCTG TGGGAGTCCT CTAGAGAGAT GAGAAGCCAG
GAGGCCTGCA
CCAAATGTCA GAAGCTTGGC ATGACCTCAT TCCGGCCACA
TCATTCTGTG
TCTCTGCATC CATTTGAACA CATTATTAAG CACCGATAAT
AGGTAGCCTG
CTGTGGGGTA TACAGCATTG ACTCAGATAT AGATCCTGAG
CTCACAGAGT
TTATAGTTAA AAAAACAAAC AGAAACACAA ACAATTTGGA
TCAAAAGGAG
AAATGATAAG TGACAAAAGC AGCACAAGGA ATTTCCCTGT
GTGGATGCTG
AGCTGTGATG GCGGGCACTG GGTACCCAAG TGAAGGTTCC
CGAGGACATG
AGTCTGTAGG AGCAAGGGCA CAAACTGCAG CTGTGAGTGC
GTGTGTGTGA
TTTGGTGTAG GTAGGTCTGT TTGCCACTTG ATGGGGCCTG
GGTTTGTTCC
TGGGGCTGGA ATGCTGGGTA TGCTCTGTGA CAAGGCTACG
CTGACAATCA
GTTAAACACA CCGGAGAAGA ACCATTTACA TGCACCTTAT
ATTTCTGTGT
ACACATCTAT TCTCAAAGCT AAAGGGTATG AAAGTGCCTG
CCTTGTTTAT
AGCCACTTGT GAGTAAAAAT TTTTTTGCAT TTTCACAAAT
TATACTTTAT
ATAAGGCATT CCACACCTAA GAACTAGTTT TGGGAAATGT
AGCCCTGGGT
TTAATGTCAA ATCAAGGCAA AAGGAATTAA ATAATGTACT
TTTGGCTAGA
GGGGTAAACT TTTTTGGCCT TTTTCTGGGG AAAATAATGT
GGGGGTGTGG.

The above-exemplified isolated DNA molecule, shown in FIGS. 4A-C and set forth as SEQ ID NO:17, contains an open reading frame from nucleotide 104 to nucleotide 1522, with a “TGA” termination codon from nucleotides 1523-1525. This open reading frame encodes a human nNR7-1 receptor, a 473 amino acid protein shown in FIGS. 5A-C and FIG. 6 and as set forth in SEQ ID NO:18.

Therefore, the present invention also relates to a substantially purified form of the novel nuclear trans-acting receptor protein, nNR7-1, which is shown in FIGS. 5A-C and FIG. 6 and as set forth in SEQ ID NO: 18, disclosed as follows:

(SEQ ID NO:18)
MTVTRTHHFK EGSLRAPAIP LHSAAAESAS NHPRGPEANL
EVRPKESWNH
ADFVHCEDTE SVPGKPSVNA DEEVGGPQIC RVCGDKATGY
HFNVMTCEGC
KGFFRRAMKR NARLRCPFRK GACEITRKTR RQCQACRLRK
CLESGMKKEM
IMSDEAVEER RALIKRKKSE RTGTQPLGVQ GLTEEQRMMI
RELMDAQMKT
FDTTFSHFKN FRLPGVLSSG CELPESLQAP SREEAAKWSQ
VRKDLCSLKV
SLQLRGEDGS VWNYKPPADS GGKEIFSLLP HMADMSTYMF
KGIISFAKVI
SYFRDLPIED QISLLKGAAF ELCQLRFNTV FNAETGTWEC
GRLSYCLEDT
AGGFQQLLLE PMLKFHYMLK KLQLHEEEYV LMQAISLFSP
DRPGVLQHRV
VDQLQEQFAI TLKSYIECNR PQPAHRFLFL KIMAMLTELR
SINAQHTQRL

LRIQDIHPFA TPLMQELFGI TGS.

A comparison of the nucleotide sequences and related open reading frames of SEQ ID NO:1 and SEQ ID NO:17 which encode nNR7 (SEQ ID NO:2) and nNR7-1 (SEQ ID NO:18) reveals nucleotide sequence divergence at the 5′ end of these cDNA clones which results in different amino terminal regions for nNR7-1 compared to nNR7. More specifically, an open reading frame from nucleotide 276 to nucleotide 350 of SEQ ID NO:1 encodes the NH₂ terminal 25 amino acids of nNR7, as set forth in FIG. 3 and SEQ ID NO:2. There is no initiating methionine residue in nNR7. In constrast, an initiating codon for methionine is present in SEQ ID NO:17 (nucleotide residue 104-106) and the 5′ portion of the coding region of this cDNA clone differs from nucleotide 104 through nucleotide 198 of SEQ ID NO:17, which encodes the NH₂ terminal 32 amino acids of nNR7-1 (SEQ ID NO:18) that differ from the initial 25 amino acid residues of nNR7. In other words, nNR7 and nNR7-1 are identical from amino acid 26-466 of nNR7 and amino acid 33-473 of nNR7-1.

The present invention also relates to biologically functional derivatives and/or mutants of nNR7 as set forth as SEQ ID NO: 18, including but not necessarily limited to amino acid substitutions, deletions, additions, amino terminal truncations and carboxy-terminal truncations such that these mutations provide for proteins or protein fragments of diagnostic, therapeutic or prophylactic use and would be useful for screening for agonists and/or antagonists of nNR7-1 function. As with many proteins, it is possible to modify many of the amino acids of nNR7 or nNR7-1, particularly those which are not found in the DNA binding domain, and still retain substantially the same biological activity as the original receptor. Thus this invention includes modified nNR7 and nNR7-1 polypeptides which have amino acid deletions, additions, or substitutions but that still retain substantially the same biological activity as nNR7 and nNR7-1. It is generally accepted that single amino acid substitutions do not usually alter the biological activity of a protein (see, e.g., Molecular Biology of the Gene, Watson et al., 1987, Fourth Ed., The Benjamin/Cummings Publishing Co., Inc., page 226; and Cunningham & Wells, 1989, Science 244:1081-1085). Accordingly, the present invention includes polypeptides where one amino acid substitution has been made in SEQ.ID.NO.:2 or SEQ ID NO:18 wherein the polypeptides still retain substantially the same biological activity as nNR7 or nNR7-1. The present invention also includes polypeptides where two or more amino acid substitutions have been made in SEQ.ID.NO.:2 or SEQ ID NO:18 wherein the polypeptides still retain substantially the same biological activity as the wild type protein. In particular, the present invention includes embodiments where the above-described substitutions are conservative substitutions. In particular, the present invention includes embodiments where the above-described substitutions do not occur in the DNA-binding domain of nNR7 or nNR7-1. When deciding which amino acid residues of nNR7 or nNR7-1 may be substituted to produce polypeptides that are functional equivalents of nNR7 or nNR7-1, one skilled in the art would be guided by a comparison of the amino acid sequence of nNR7 or nNR7-1 with the amino acid sequences of related proteins, One skilled in the art would also recognize that polypeptides that are functional equivalents of nNR7 or nNR7-1 and have changes from the amino acid sequence of these respective proteins that are small deletions or insertions of amino acids could also be produced by following the same guidelines, (i.e, minimizing the differences in amino acid sequence between nNR7 or nNR7-1 and related proteins. Small deletions or insertions are generally in the range of about 1 to 5 amino acids. The effect of such small deletions or insertions on the biological activity of the modified nNR7 or nNR7-1 polypeptide can easily be assayed by producing the polypeptide synthetically or by making the required changes in DNA encoding nNR7 or nNR7-1 and then expressing the DNA recombinantly and assaying the protein produced by such recombinant expression.

The present invention also relates to isolated nucleic acid molecules which are fusion constructions expressing fusion proteins useful in assays to identify compounds which modulate wild-type human nNR7 and/or nNR7-1 activity. A preferred aspect of this portion of the invention includes, but is not limited to, glutathione S-transferase GST-nNR7 and/or GST-nNR7-1 fusion constructs. These fusion constructs include, but are not limited to, all or a portion of the ligand-binding domain of nNR7 and/or nNR7-1, respectively, as an in-frame fusion at the carboxy terminus of the GST gene. The disclosure of SEQ ID Nos:1, 2, 17 and 18 allow the artisan of ordinary skill to construct any such nucleic acid molecule encoding a GST-nuclear receptor fusion protein. Soluble recombinant GST-nuclear receptor fusion proteins may be expressed in various expression systems, including Spodoptera frugiperda (Sf21) insect cells (Invitrogen) using a baculovirus expression vector (e.g., Bac-N-Blue DNA from Invitrogen or pAcG2T from Pharmingen).

It is also within the purview of the artisan of ordinary skill to use the DNA molecules of the present invention to construct DNA expression vectors for use in transactivation assays to identify modulators of nNR7, nNR7-1 and/or DNA molecules or proteins which interact with nNR7-like proteins. For example, a portion of SEQ ID NO:1 or SEQ ID NO:17 may be fused downstream of an active promoter, such as a CMV promoter fragment, so as to overexpress a receptor protein. A second construct includes regulatory regions from the human CYP3A4 gene (such as the human CYP3A4 promoter region or a response element [e.g., SEQ ID NO:24]) fused to a reporter gene, such as SEAP (secreted placental alkaline phosphatase, LacZ or chloremphenicol acetly transferase [CAT]). These constructs may then be utilized in a transactivation assay to identify modulators of CYP3A4 levels in vivo. It will be known that various modifications to either or both the receptor construct and/or the reporter construct may be made without effecting the effectiveness of the transactivation assay. For example, the native nNR7 or nNR7-1 promoter may be utilized instead of a hybrid promoter such as the CMV promoter. Or it may be useful to use a different reporter gene, such as LacZ.

Therefore, the present invention is also directed to methods for screening for compounds which modulate the expression of DNA or RNA encoding a human nNR7 and/or nNR7-1 protein. Compounds which modulate these activities may be DNA, RNA, peptides, proteins, or non-proteinaceous organic molecules. Compounds may modulate by increasing or attenuating the expression of DNA or RNA encoding human nNR7 and/or nNR7-1, or the function of human nNR7 and/or nNR7-1. Compounds that modulate the expression of DNA or RNA encoding human nNR7 and/or nNR7-1 or the biological function thereof may be detected by a variety of assays. The assay may be a simple “yes/no” assay to determine whether there is a change in expression or function. The assay may be made quantitative by comparing the expression or function of a test sample with the levels of expression or function in a standard sample. Kits containing human nNR7 and/or nNR7-1, antibodies to human nNR7 and/or nNR7-1, or modified human nNR7 and/or nNR7-1 may be prepared by known methods for such uses.

The DNA molecules, RNA molecules, recombinant protein and antibodies of the present invention may be used to screen and measure levels of human nNR7 and/or nNR7-1. The recombinant proteins, DNA molecules, RNA molecules and antibodies lend themselves to the formulation of kits suitable for the detection and typing of human nNR7 and/or nNR7-1. Such a kit would comprise a compartmentalized carrier suitable to hold in close confinement at least one container. The carrier would further comprise reagents such as recombinant nNR7 and/or nNR7-1 or anti-nNR7 and/or nNR7-1 antibodies suitable for detecting human nNR7 and/or nNR7-1. The carrier may also contain a means for detection such as labeled antigen or enzyme substrates or the like.

Pharmaceutically useful compositions comprising modulators of human nNR7 and/or nNR7-1 may be formulated according to known methods such as by the admixture of a pharmaceutically acceptable carrier. Examples of such carriers and methods of formulation may be found in Remington's Pharmaceutical Sciences. To form a pharmaceutically acceptable composition suitable for effective administration, such compositions will contain an effective amount of the protein, DNA, RNA, modified human nNR7 and/or nNR7-1, or either nNR7 and/or nNR7-1 agonists or antagonists.

Therefore, the present invention includes assays by which modulators of cytochrome P450 enzymes, especially CYP 450 enzymes which comprise the response element 5′-TGAACTCAAAGGAGGTCA-3′ (SEQ ID NO:24), and especially human CYP3A4. Methods for identifying agonists and antagonists of other receptors are well known in the art and can be adapted to identify compounds which effect in vivo levels of CYP3A4. Accordingly, the present invention includes a method for determining whether a substance is a potential modulator of CYP3A4 levels that comprises:

(a) transfecting or transforming cells with an expression vector encoding nNR7 or nNR7-1, also known as the receptor vector;

(b) transfecting or transforming the cells of step (a) with second expression vector which comprises a response element known to bind nNR7 or nNR7-1 and a promoter fragment fused upstream of a reporter gene, also known as a reporter vector.

(c) allowing the transfected cells to grow for a time sufficient to allow nNR7 or nNR7-1 to be expressed;

(d) exposing the test cells to a substance while not exposing control cells to the test substance;

(e) measuring the expression of the reporter gene in both the test cells and control cells; where if the amount of binding of expression in the test cells is greater than in the control cells, the substance may enhance or act as an agonist to CYP3A4 activity, whereas the opposite effect may suggest a possible antagonist of CYP3A4 activity.

The conditions under which step (d) of the method is practiced are conditions that are typically used in the art for the study of protein-ligand interactions: e.g., physiological pH; salt conditions such as those represented by such commonly used buffers as PBS or in tissue culture media; a temperature of about 4° C. to about 55° C.

Alternatively, the transactivation assay may be carried out as follows: (a) provide test cells by transfecting cells with a receptor expression vector that directs the expression of nNR7 or nNR7-1 in the cells;

(b) providing test cells by transfecting the cells of step (a) with a second reporter expression vector that directs expression of a reporter gene under control of a regulatory element which is responsive to nNR7 or nNR7-1 (b) exposing the test cells to the substance;

(c) measuring the amount of binding of expression of the reporter gene;

(d) comparing the amount of expression of the reporter gene in the test cells with the amount of expression of the reporter gene in control cells that has been transfected with a reporter vector of step (b) but not a receptor vector of step (a).

Therefore, it is evident that any number of variations known to one of skill in the art may be utilized in order to provide for an assay to measure the effect of a substance on the ability of the nuclear receptor proteins of the present invention to effect transcription of a promoter of interest (e.g., effecting CYP3A4 gene expression).

The present invention also includes a method for determining whether a substance is capable of binding to nNR7 and/or nNR7-1, i.e., whether the substance is a potential agonist or an antagonist of nNR7 and/or nNR7-1, where the method comprises:

(a) providing test cells by transfecting cells with an expression vector that directs the expression of nNR7 and/or nNR7-1 in the cells;

(b) exposing the test cells to the substance;

(c) measuring the amount of binding of the substance to nNR7 and/or nNR7-1;

(d) comparing the amount of binding of the substance to nNR7 or nNR7-1 in the test cells with the amount of binding of the substance to control cells that have not been transfected with nNR7 or nNR7-1; wherein if the amount of binding of the substance is greater in the test cells as compared to the control cells, the substance is capable of binding to nNR7 or nNR7-1. Determining whether the substance is actually an agonist or antagonist can then be accomplished by the use of functional assays such as the transactivation assay as described above.

The conditions under which step (b) of the method is practiced are conditions that are typically used in the art for the study of protein-ligand interactions: e.g., physiological pH; salt conditions such as those represented by such commonly used buffers as PBS or in tissue culture media; a temperature of about 4° C. to about 55° C.

The assays described above can be carried out with cells that have been transiently or stably transfected with nNR7 or nNR7-1. Transfection is meant to include any method known in the art for introducing nNR7 or nNR7-1 into the test cells. For example, transfection includes calcium phosphate or calcium chloride mediated transfection, lipofection, infection with a retroviral construct containing nNR7 or nNR7-1, and electroporation.

Where binding of the substance or agonist to nNR7 or nNR7-1 is measured, such binding can be measured by employing a labeled substance or agonist. The substance or agonist can be labeled in any convenient manner known to the art, e.g., radioactively, fluorescently, enzymatically.

In particular embodiments of the above-described methods, nNR7 or nNR7-1 has an amino acid sequence of SEQ ID NO:2.

The isolated nucleic acid molecule of the present invention may include a deoxyribonucleic acid molecule (DNA), such as genomic DNA and complementary DNA (cDNA), which may be single (coding or noncoding strand) or double stranded, as well as synthetic DNA, such as a synthesized, single stranded polynucleotide. The isolated nucleic acid molecule of the present invention may also include a ribonucleic acid molecule (RNA).

It is known that there is a substantial amount of redundancy in the various codons which code for specific amino acids. Therefore, this invention is also directed to those DNA sequences encode RNA comprising alternative codons which code for the eventual translation of the identical amino acid, as shown below:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His=Histidine: codons CAC, CAU

I=Ile=Isoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asp=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Therefore, the present invention discloses codon redundancy which may result in differing DNA molecules expressing an identical protein. For purposes of this specification, a sequence bearing one or more replaced codons will be defined as a degenerate variation. Also included within the scope of this invention are mutations either in the DNA sequence or the translated protein which do not substantially alter the ultimate physical properties of the expressed protein. For example, substitution of valine for leucine, arginine for lysine, or asparagine for glutamine may not cause a change in functionality of the polypeptide.

It is known that DNA sequences coding for a peptide may be altered so as to code for a peptide having properties that are different than those of the naturally occurring peptide. Methods of altering the DNA sequences include but are not limited to site directed mutagenesis. Examples of altered properties include but are not limited to changes in the affinity of an enzyme for a substrate or a receptor for a ligand.

As used herein, “purified” and “isolated” are utilized interchangeably to stand for the proposition that the nucleic acid, protein, or respective fragment thereof in question has been substantially removed from its in vivo environment so that it may be manipulated by the skilled artisan, such as but not limited to nucleotide sequencing, restriction digestion, site-directed mutagenesis, and subcloning into expression vectors for a nucleic acid fragment as well as obtaining the protein or protein fragment in pure quantities so as to afford the opportunity to generate polyclonal antibodies, monoclonal antibodies, amino acid sequencing, and peptide digestion. Therefore, the nucleic acids claimed herein may be present in whole cells or in cell lysates or in a partially purified or substantially purified form. A nucleic acid is considered substantially purified when it is purified away from environmental contaminants. Thus, a nucleic acid sequence isolated from cells is considered to be substantially purified when purified from cellular components by standard methods while a chemically synthesized nucleic acid sequence is considered to be substantially purified when purified from its chemical precursors.

The present invention also relates to recombinant vectors and recombinant hosts, both prokaryotic and eukaryotic, which contain the substantially purified nucleic acid molecules disclosed throughout this specification.

Therefore, the present invention also relates to methods of expressing nNR7 and/or nNR7-1 and biological equivalents disclosed herein, assays employing these recombinantly expressed gene products, cells expressing these gene products, and agonistic and/or antagonistic compounds identified through the use of assays utilizing these recombinant forms, including, but not limited to, one or more modulators of the human nNR7 and/or nNR7-1 either through direct contact LBD or through direct or indirect contact with a ligand which either interacts with the DBD or with the wild-type transcription complex which nNR7 and/or nNR7-1 interacts in trans, thereby modulating cell differentiation or cell development.

As used herein, a “biologically functional derivative” of a wild-type human nNR7 and/or nNR7-1 possesses a biological activity that is related to the biological activity of the wild type human nNR7 and/or nNR7-1. The term “functional derivative” is intended to include the “fragments,” “mutants,” “variants,” “degenerate variants,” “analogs” and “homologues” of the wild type human nNR7 and/or nNR-1 protein. The term “fragment” is meant to refer to any polypeptide subset of wild-type human nNR7 and/or nNR7-1, including but not necessarily limited to amino acid substitutions, deletions, additions, amino terminal truncations and carboxy-terminal truncations. The term “mutant” is meant to refer a subset of a biologically active fragment that may be substantially similar to the wild-type form but possesses distinguishing biological characteristics. Such altered characteristics include but are in no way limited to altered substrate binding, altered substrate affinity and altered sensitivity to chemical compounds affecting biological activity of the human nNR7 and/or nNR7-1 or human nNR7 and/or nNR7-1 functional derivative. The term “variant” is meant to refer to a molecule substantially similar in structure and function to either the entire wild-type protein or to a fragment thereof. A molecule is “substantially similar” to a wild-type human nNR7 and/or nNR7-1-like protein if both molecules have substantially similar structures or if both molecules possess similar biological activity. Therefore, if the two molecules possess substantially similar activity, they are considered to be variants even if the structure of one of the molecules is not found in the other or even if the two amino acid sequences are not identical. The term “analog” refers to a molecule substantially similar in function to either the full-length human nNR7 and/or nNR7-1 protein or to a biologically functional derivative thereof.

Any of a variety of procedures may be used to clone human nNR7 and/or nNR7-1. These methods include, but are not limited to, (1) a RACE PCR cloning technique (Frohman, et al., 1988, Proc. Natl. Acad. Sci. USA 85: 8998-9002). 5¢ and/or 3¢ RACE may be performed to generate a full-length cDNA sequence. This strategy involves using gene-specific oligonucleotide primers for PCR amplification of human nNR7 and/or nNR7-1 cDNA. These gene-specific primers are designed through identification of an expressed sequence tag (EST) nucleotide sequence which has been identified by searching any number of publicly available nucleic acid and protein databases; (2) direct functional expression of the human nNR7 and/or nNR7-1 cDNA following the construction of a human nNR7 and/or nNR7-1-containing cDNA library in an appropriate expression vector system; (3) screening a human nNR7 and/or nNR7-1-containing cDNA library constructed in a bacteriophage or plasmid shuttle vector with a labeled degenerate oligonucleotide probe designed from the amino acid sequence of the human nNR7 and/or nNR7-1 protein; (4) screening a human nNR7 and/or nNR7-1-containing cDNA library constructed in a bacteriophage or plasmid shuttle vector with a partial cDNA encoding the human nNR7 and/or nNR7-1 protein. This partial cDNA is obtained by the specific PCR amplification of human nNR7 and/or nNR7-1 DNA fragments through the design of degenerate oligonucleotide primers from the amino acid sequence known for other kinases which are related to the human nNR7 and/or nNR7-1 protein; (5) screening a human nNR7 and/or nNR7-1-containing cDNA library constructed in a bacteriophage or plasmid shuttle vector with a partial cDNA encoding the human nNR7 and/or nNR7-1 protein. This strategy may also involve using gene-specific oligonucleotide primers for PCR amplification of human nNR7 and/or nNR7-1 cDNA identified as an EST as described above; or (6) designing 5¢ and 3¢ gene specific oligonucleotides using SEQ ID NO: 1 as a template so that either the full-length cDNA may be generated by known PCR techniques, or a portion of the coding region may be generated by these same known PCR techniques to generate and isolate a portion of the coding region to use as a probe to screen one of numerous types of cDNA and/or genomic libraries in order to isolate a full-length version of the nucleotide sequence encoding human nNR7 and/or nNR7-1.

It is readily apparent to those skilled in the art that other types of libraries, as well as libraries constructed from other cell types-or species types, may be useful for isolating a nNR7 and/or nNR7-1-encoding DNA or a nNR7 and/or nNR7-1 homologue. Other types of libraries include, but are not limited to, cDNA libraries derived from other cells or cell lines other than human cells or tissue such as murine cells, rodent cells or any other such vertebrate host which may contain nNR7 and/or nNR7-1-encoding DNA. Additionally a nNR7 and/or nNR7-1 gene and homologues may be isolated by oligonucleotide- or polynucleotide-based hybridization screening of a vertebrate genomic library, including but not limited to, a murine genomic library, a rodent genomic library, as well as concomitant human genomic DNA libraries.

It is readily apparent to those skilled in the art that suitable cDNA libraries may be prepared from cells or cell lines which have nNR7 and/or nNR7-1 activity. The selection of cells or cell lines for use in preparing a cDNA library to isolate a cDNA encoding nNR7 and/or nNR7-1 may be done by first measuring cell-associated nNR7 and/or nNR7-1 activity using any known assay available for such a purpose.

Preparation of cDNA libraries can be performed by standard techniques well known in the art. Well known cDNA library construction techniques can be found for example, in Sambrook et al., 1989, Molecular Cloning: A Laboratory Manual; Cold Spring Harbor Laboratory, Cold Spring Harbor, N.Y. Complementary DNA libraries may also be obtained from numerous commercial sources, including but not limited to Clontech Laboratories, Inc. and Stratagene.

It is also readily apparent to those skilled in the art that DNA encoding human nNR7 and/or nNR7-1 may also be isolated from a suitable genomic DNA library. Construction of genomic DNA libraries can be performed by standard techniques well known in the art. Well known genomic DNA library construction techniques can be found in Sambrook, et al., supra.

In order to clone the human nNR7 and/or nNR7-1 gene by one of the preferred methods, the amino acid sequence or DNA sequence of human nNR7 and/or nNR7-1 or a homologous protein may be necessary. To accomplish this, the nNR7 and/or nNR7-1 protein or a homologous protein may be purified and partial amino acid sequence determined by automated sequenators. It is not necessary to determine the entire amino acid sequence, but the linear sequence of two regions of 6 to 8 amino acids can be determined for the PCR amplification of a partial human nNR7 and/or nNR7-1 DNA fragment. Once suitable amino acid sequences have been identified, the DNA sequences capable of encoding them are synthesized. Because the genetic code is degenerate, more than one codon may be used to encode a particular amino acid, and therefore, the amino acid sequence can be encoded by any of a set of similar DNA oligonucleotides. Only one member of the set will be identical to the human nNR7 and/or nNR7-1 sequence but others in the set will be capable of hybridizing to human nNR7 and/or nNR7-1 DNA even in the presence of DNA oligonucleotides with mismatches. The mismatched DNA oligonucleotides may still sufficiently hybridize to the human nNR7 and/or nNR7-1 DNA to permit identification and isolation of human nNR7 and/or nNR7-1 encoding DNA. Alternatively, the nucleotide sequence of a region of an expressed sequence may be identified by searching one or more available genomic databases. Gene-specific primers may be used to perform PCR amplification of a cDNA of interest from either a cDNA library or a population of cDNAs. As noted above, the appropriate nucleotide sequence for use in a PCR-based method may be obtained from SEQ ID NO: 1, either for the purpose of isolating overlapping 5¢ and 3¢ RACE products for generation of a full-length sequence coding for human nNR7 and/or nNR7-1, or to isolate a portion of the nucleotide sequence coding for human nNR7 and/or nNR7-1 for use as a probe to screen one or more cDNA- or genomic-based libraries to isolate a full-length sequence encoding human nNR7 and/or nNR7-1-like proteins.

In one exemplified method, the human nNR7 full-length cDNA of the present invention were generated by screening a mixed cDNA library with DNA fragments corresponding to human AR, ERb, GR and VDR DNA binding domain regions. Positive clones were excised from lZAPII phages and the plasmid DNA was sequenced directly M13 forward and reverse primers. A Blast search of the sequence information was obtained using computer program PhredPhrap. A single cDNA clone was shown to comprise the open reading frame of a novel human nuclear receptor. DNA sequencing was continued from both 5′ and 3′ ends on nNR7, which identified the presence of an intron. A cDNA fragment flanking the intron region was retrieved via PCR using a first oligonucleotide primer primer pair and double nested primers. A prominent DNA fragment of ˜1.5 kb was amplified from testis and brain cDNAs. The fragment was purified and subcloned into pCRII vector (Invitrogen, San Diego, Calif.). Automated sequencing was performed on several of the clones. The complete sequencing revealed a 3.1 kb cDNA which codes an authentic novel nuclear receptor, nNR7.

In a second exemplified method oligo primers were used to amplify nNR7 5′ cDNA ends from human liver cDNA. DNA product from one round PCR reaction was gel purified, subcloned into a plasmid vector and subjected to nucleotide sequencing. Ambiguities and/or discrepancies between automated base calling in sequencing reads were visually examined and edited to the correct base call. One clone was found to have an open reading frame differing from the open reading frame disclosed for SEQ ID NO:1, such that this additional exemplifed cDNA (as disclosed in SEQ ID NO:17) contains an in frame Met and up-stream stop codons. The site of divergence (as discussed herein) is the same as the other two additional clones derived from 5′-RACE (rapid amplification of cDNA ends). The cDNA clone disclosed in FIGS. 4A-4C and set forth as SEQ ID NO:17 is a complementary DNA sequence to a completely processed mRNA which expresses an especially preferred nuclear receptor protein of the present invention (i.e, nNR7-1).

A variety of mammalian expression vectors may be used to express recombinant human nNR7 and/or nNR7-1 in mammalian cells. Expression vectors are defined herein as DNA sequences that are required for the transcription of cloned DNA and the translation of their mRNAs in an appropriate host. Such vectors can be used to express eukaryotic DNA in a variety of hosts such as bacteria, blue green algae, plant cells, insect cells and animal cells. Specifically designed vectors allow the shuttling of DNA between hosts such as bacteria-yeast or bacteria-animal cells. An appropriately constructed expression vector should contain: an origin of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one which causes mRNAs to be initiated at high frequency. Expression vectors may include, but are not limited to, cloning vectors, modified cloning vectors, specifically designed plasmids or viruses.

Commercially available mammalian expression vectors which may be suitable for recombinant human nNR7 and/or nNR7-1 expression, include but are not limited to pcDNA3.1 (Invitrogen), pLITMUS28, pLITMUS29, pLITMUS38 and pLITMUS39 (New England Bioloabs), pcDNAI, pcDNAIamp (Invitrogen), pcDNA3 (Invitrogen), pMC1neo (Stratagene), pXT1 (Stratagene), pSG5 (Stratagene), EBO-pSV2-neo (ATCC 37593) pBPV-1(8-2) (ATCC 37110), pdBPV-MMTneo (342-12) (ATCC 37224), pRSVgpt (ATCC 37199), pRSVneo (ATCC 37198), pSV2-dhfr (ATCC 37146), pUCTag (ATCC 37460), and lZD35 (ATCC 37565).

A variety of bacterial expression vectors may be used to express recombinant human nNR7 and/or nNR7-1 in bacterial cells. Commercially available bacterial expression vectors which may be suitable for recombinant human nNR7 and/or nNR7-1 expression include, but are not limited to pCRII (Invitrogen), pCR2.1 (Invitrogen), pQE (Qiagen), pET11a (Novagen), lambda gt11 (Invitrogen), and pKK223-3 (Pharmacia).

A variety of fungal cell expression vectors may be used to express recombinant human nNR7 and/or nNR7-1 in fungal cells. Commercially available fungal cell expression vectors which may be suitable for recombinant human nNR7 and/or nNR7-1 expression include but are not limited to pYES2 (Invitrogen) and Pichia expression vector (Invitrogen).

A variety of insect cell expression vectors may be used to express recombinant receptor in insect cells. Commercially available insect cell expression vectors which may be suitable for recombinant expression of human nNR7 and/or nNR7-1 include but are not limited to pBlueBacIII and pBlueBacHis2 (Invitrogen), and pAcG2T (Pharmingen).

An expression vector containing DNA encoding a human nNR7 and/or nNR7-1-like protein may be used for expression of human nNR7 and/or nNR7-1 in a recombinant host cell. Recombinant host cells may be prokaryotic or eukaryotic, including but not limited to bacteria such as E. coli, fungal cells such as yeast, mammalian cells including but not limited to cell lines of human, bovine, porcine, monkey and rodent origin, and insect cells including but not limited to Drosophila- and silkworm-derived cell lines. Cell lines derived from mammalian species which may be suitable and which are commercially available, include but are not limited to, L cells L-M(TK⁻) (ATCC CCL 1.3), L cells L-M (ATCC CCL 1.2), Saos-2 (ATCC HTB-85), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171) and CPAE (ATCC CCL 209).

The expression vector may be introduced into host cells via any one of a number of techniques including but not limited to transformation, transfection, protoplast fusion, and electroporation. The expression vector-containing cells are individually analyzed to determine whether they produce human nNR7 and/or nNR7-1 protein. Identification of human nNR7 and/or nNR7-1 expressing cells may be done by several means, including but not limited to immunological reactivity with anti-human nNR7 and/or nNR7-1 antibodies, labeled ligand binding and the presence of host cell-associated human nNR7 and/or nNR7-1 activity.

The cloned human nNR7 and/or nNR7-1 cDNA obtained through the methods described above may be recombinantly expressed by molecular cloning into an expression vector (such as pcDNA3.1, pQE, pBlueBacHis2 and pLITMUS28) containing a suitable promoter and other appropriate transcription regulatory elements, and transferred into prokaryotic or eukaryotic host cells to produce recombinant human nNR7 and/or nNR7-1. Techniques for such manipulations can be found described in Sambrook, et al., supra , are discussed at length in the Example section and are well known and easily available to the artisan of ordinary skill in the art.

Expression of human nNR7 and/or nNR7-1 DNA may also be performed using in vitro produced synthetic mRNA. Synthetic mRNA can be efficiently translated in various cell-free systems, including but not limited to wheat germ extracts and reticulocyte extracts, as well as efficiently translated in cell based systems, including but not limited to microinjection into frog oocytes, with microinjection into frog oocytes being preferred.

To determine the human nNR7 and/or nNR7-1 cDNA sequence(s) that yields optimal levels of human nNR7 and/or nNR7-1, cDNA molecules including but not limited to the following can be constructed: a cDNA fragment containing the full-length open reading frame for human nNR7 and/or nNR7-1 as well as various constructs containing portions of the cDNA encoding only specific domains of the protein or rearranged domains of the protein. All constructs can be designed to contain none, all or portions of the 5¢ and/or 3¢ untranslated region of a human nNR7 and/or nNR7-1 cDNA. The expression levels and activity of human nNR7 and/or nNR7-1 can be determined following the introduction, both singly and in combination, of these constructs into appropriate host cells. Following determination of the human nNR7 and/or nNR7-1 cDNA cassette yielding optimal expression in transient assays, this nNR7 and/or nNR7-1 cDNA construct is transferred to a variety of expression vectors (including recombinant viruses), including but not limited to those for mammalian cells, plant cells, insect cells, oocytes, bacteria, and yeast cells.

The present invention also relates to polyclonal and monoclonal antibodies raised in response to either the human form of nNR7 and/or nNR7-1 disclosed herein, or a biologically functional derivative thereof. It will be especially preferable to raise antibodies against epitopes within the NH₂-terminal domain of nNR7 and/or nNR7-1, which show the least homology to other known proteins belonging to the human nuclear receptor superfamily.

Recombinant nNR7 and/or nNR7-1 protein can be separated from other cellular proteins by use of an immunoaffinity column made with monoclonal or polyclonal antibodies specific for full-length nNR7 and/or nNR7-1 protein, or polypeptide fragments of nNR7 and/or nNR7-1 protein. Additionally, polyclonal or monoclonal antibodies may be raised against a synthetic peptide (usually from about 9 to about 25 amino acids in length) from a portion of the protein as disclosed in SEQ ID NO: 2 or SEQ ID NO:18. Monospecific antibodies to human nNR7 and/or nNR7-1 are purified from mammalian antisera containing antibodies reactive against human nNR7 and/or nNR7-1 or are prepared as monoclonal antibodies reactive with human nNR7 and/or nNR7-1 using the technique of Kohler and Milstein (1975, Nature 256: 495-497). Monospecific antibody as used herein is defined as a single antibody species or multiple antibody species with homogenous binding characteristics for human nNR7 and/or nNR7-1. Homogenous binding as used herein refers to the ability of the antibody species to bind to a specific antigen or epitope, such as those associated with human nNR7 and/or nNR7-1, as described above. Human nNR7 and/or nNR7-1-specific antibodies are raised by immunizing animals such as mice, rats, guinea pigs, rabbits, goats, horses and the like, with an appropriate concentration of human nNR7 and/or nNR7-1 protein or a synthetic peptide generated from a portion of human nNR7 and/or nNR7-1 with or without an immune adjuvant.

Preimmune serum is collected prior to the first immunization. Each animal receives between about 0.1 mg and about 1000 mg of human nNR7 and/or nNR7-1 protein associated with an acceptable immune adjuvant. Such acceptable adjuvants include, but are not limited to, Freund's complete, Freund's incomplete, alum-precipitate, water in oil emulsion containing Corynebacterium parvum and tRNA. The initial immunization consists of human nNR7 and/or nNR7-1 protein or peptide fragment thereof in, preferably, Freund's complete adjuvant at multiple sites either subcutaneously (SC), intraperitoneally (IP) or both. Each animal is bled at regular intervals, preferably weekly, to determine antibody titer. The animals may or may not receive booster injections following the initial immunization. Those animals receiving booster injections are generally given an equal amount of human nNR7 and/or nNR7-1 in Freund's incomplete adjuvant by the same route. Booster injections are given at about three week intervals until maximal titers are obtained. At about 7 days after each booster immunization or about weekly after a single immunization, the animals are bled, the serum collected, and aliquots are stored at about −20° C.

Monoclonal antibodies (mAb) reactive with human nNR7 and/or nNR7-1 are prepared by immunizing inbred mice, preferably Balb/c, with human nNR7 and/or nNR7-1 protein. The mice are immunized by the IP or SC route with about 1 mg to about 100 mg, preferably about 10 mg, of human nNR7 and/or nNR7-1 protein in about 0.5 ml buffer or saline incorporated in an equal volume of an acceptable adjuvant, as discussed above. Freund's complete adjuvant is preferred. The mice receive an initial immunization on day 0 and are rested for about 3 to about 30 weeks. Immunized mice are given one or more booster immunizations of about 1 to about 100 mg of human nNR7 and/or nNR7-1 in a buffer solution such as phosphate buffered saline by the intravenous (IV) route. Lymphocytes, from antibody positive mice, preferably splenic lymphocytes, are obtained by removing spleens from immunized mice by standard procedures known in the art. Hybridoma cells are produced by mixing the splenic lymphocytes with an appropriate fusion partner, preferably myeloma cells, under conditions which will allow the formation of stable hybridomas. Fusion partners may include, but are not limited to: mouse myelomas P3/NS1/Ag 4-1, MPC-11, S-194 and Sp 2/0, with Sp 2/0 being preferred. The antibody producing cells and myeloma cells are fused in polyethylene glycol, about 1000 mol. wt., at concentrations from about 30% to about 50%. Fused hybridoma cells are selected by growth in hypoxanthine, thymidine and aminopterin supplemented Dulbecco's Modified Eagles Medium (DMEM) by procedures known in the art. Supernatant fluids are collected form growth positive wells on about days 14, 18, and 21 and are screened for antibody production by an immunoassay such as solid phase immunoradioassay (SPIRA) using human nNR7 and/or nNR7-1 as the antigen. The culture fluids are also tested in the Ouchterlony precipitation assay to determine the isotype of the mAb. Hybridoma cells from antibody positive wells are cloned by a technique such as the soft agar technique of MacPherson, 1973, Soft Agar Techniques, in Tissue Culture Methods and Applications, Kruse and Paterson, Eds., Academic Press.

Monoclonal antibodies are produced in vivo by injection of pristine primed Balb/c mice, approximately 0.5 ml per mouse, with about 2×10⁶ to about 6×10⁶ hybridoma cells about 4 days after priming. Ascites fluid is collected at approximately 8-12 days after cell transfer and the monoclonal antibodies are purified by techniques known in the art.

In vitro production of anti-human nNR7 and/or nNR7-1 mAb is carried out by growing the hybridoma in DMEM containing about 2% fetal calf serum to obtain sufficient quantities of the specific mAb. The mAb are purified by techniques known in the art.

Antibody titers of ascites or hybridoma culture fluids are determined by various serological or immunological assays which include, but are not limited to, precipitation, passive agglutination, enzyme-linked immunosorbent antibody (ELISA) technique and radioimmunoassay (RIA) techniques. Similar assays are used to detect the presence of human nNR7 and/or nNR7-1 in body fluids or tissue and cell extracts.

It is readily apparent to those skilled in the art that the above described methods for producing monospecific antibodies may be utilized to produce antibodies specific for human nNR7 and/or nNR7-1 peptide fragments, or full-length human nNR7 and/or nNR7-1.

Human nNR7 and/or nNR7-1 antibody affinity columns are made, for example, by adding the antibodies to Affigel-10 (Biorad), a gel support which is pre-activated with N-hydroxysuccinimide esters such that the antibodies form covalent linkages with the agarose gel bead support. The antibodies are then coupled to the gel via amide bonds with the spacer arm. The remaining activated esters are then quenched with 1M ethanolamine HCl (pH 8.0). The column is washed with water followed by 0.23 M glycine HCl (pH 2.6) to remove any non-conjugated antibody or extraneous protein. The column is then equilibrated in phosphate buffered saline (pH 7.3) and the cell culture supernatants or cell extracts containing full-length human nNR7 and/or nNR7-1 or human nNR7 and/or nNR7-1 protein fragments are slowly passed through the column. The column is then washed with phosphate buffered saline until the optical density (A280) falls to background, then the protein is eluted with 0.23 M glycine-HCl (pH 2.6). The purified human nNR7 and/or nNR7-1 protein is then dialyzed against phosphate buffered saline.

Levels of human nNR7 and/or nNR7-1 in host cells is quantified by a variety of techniques including, but not limited to, immunoaffinity and/or ligand affinity techniques. nNR7 and/or nNR7-1-specific affinity beads or nNR7 and/or nNR7-1-specific antibodies are used to isolate ³⁵S-methionine labeled or unlabelled nNR7 and/or nNR7-1. Labeled nNR7 and/or nNR7-1 protein is analyzed by SDS-PAGE. Unlabelled nNR7 and/or nNR7-1 protein is detected by Western blotting, ELISA or RIA assays employing either nNR7 and/or nNR7-1 protein specific antibodies and/or antiphosphotyrosine antibodies.

Following expression of nNR7 and/or nNR7-1 in a host cell, nNR7 and/or nNR7-1 protein may be recovered to provide nNR7 and/or nNR7-1 protein in active form. Several nNR7 and/or nNR7-1 protein purification procedures are available and suitable for use. Recombinant nNR7 and/or nNR7-1 protein may be purified from cell lysates and extracts, or from conditioned culture medium, by various combinations of, or individual application of salt fractionation, ion exchange chromatography, size exclusion chromatography, hydroxylapatite adsorption chromatography and hydrophobic interaction chromatography.

Therapeutic or diagnostic compositions of the invention are administered to an individual in amounts sufficient to treat or diagnose disorders. The effective amount may vary according to a variety of factors such as the individual's condition, weight, sex and age. Other factors include the mode of administration.

The pharmaceutical compositions may be provided to the individual by a variety of routes such as subcutaneous, topical, oral and intramuscular.

The term “chemical derivative” describes a molecule that contains additional chemical moieties which are not normally a part of the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington's Pharmaceutical Sciences.

Compounds identified according to the methods disclosed herein may be used alone at appropriate dosages. Alternatively, co-administration or sequential administration of other agents may be desirable.

The present invention also has the objective of providing suitable topical, oral, systemic and parenteral pharmaceutical formulations for use in the novel methods of treatment of the present invention. The compositions containing compounds identified according to this invention as the active ingredient can be administered in a wide variety of therapeutic dosage forms in conventional vehicles for administration. For example, the compounds can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

For combination treatment with more than one active agent, where the active agents are in separate dosage formulations, the active agents can be administered concurrently, or they each can be administered at separately staggered times.

The dosage regimen utilizing the compounds of the present invention is selected in accordance with a variety of factors including type, species, age, weight, sex and medical condition of the patient; the severity of the condition to be treated; the route of administration; the renal, hepatic and cardiovascular function of the patient; and the particular compound thereof employed. A physician or veterinarian of ordinary skill can readily determine and prescribe the effective amount of the drug required to prevent, counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug.

The following example is provided to illustrate the present invention without, however, limiting the same hereto.

### EXAMPLE 1

### Isolation and Characterization of a DNA Molecule Encoding nNR7

DNA fragments encoding DBD regions of androgen receptor (AR; Lubahn, et al., 1988, Mol. Endocrinol. 2 (12): 1265-1275), estrogen receptor b (ERb; Mosselman, et al., 1996, FEBS Lett. 392 (1): 49-53), glucocorticoid receptor (GR; Hollenberg, et al., 1985, Nature 318: 635-641) and vitamin D receptor (VDR; Baker, et al., 1988, Proc. Natl. Acad. Sci. (U.S.A) 85 (10): 3294-3298) were generated by PCR made from human fetal brain mRNA for AR, ERb and Grand human small intestine mRNA for VDR. These cDNA fragments were subcloned into pCR cloning vectors as described by the manufacturer. The following oligonucleotide primers were utilized to generate fragments the above-mentioned cDNA fragments for plasmid subcloning:

1. GR-R 5′-TTTCGAGCTTCCAGGTTCAT-3′ (SEQ ID NO: 9)

2. GR-F 5′-CTCCCAAACTCTGCCTGGTG-3′ (SEQ ID NO: 10)

3. ERB-R 5′-CGGGAGCCACACTTCACCAT-3′ (SEQ ID NO: 11)

4. ERB-F 5′-GCTCACTTCTGCGCTGTCTG-3′ (SEQ ID NO: 12)

5. AR-R 5′-TTCCGGGCTCCCAGAGTCAT-3′ (SEQ ID NO: 13)

6. AR-F 5′-CAGAAGACCTGCCTGATCTG-3′ (SEQ ID NO: 14)

7. VDR-R 5′-GAAATGAACTCCTTCATCAT-3′ (SEQ ID NO: 15)

8. VDR-F 5′-CCGGATCTGTGGGGTGTGTG-3′ (SEQ ID NO: 16).

The DNA fragments were purified using a Qiagen gel extraction kit. Phosphorylation, self-ligation and transformation of the purified DNA was carried out as recommended by the manufacturer.

These DBD probes were utilized to screen a mixed cDNA library. from adrenal, bone marrow, brain, fetal brain, heart, fetal kidney, liver, fetal liver, lung, fetal lung, mammary gland, pancreas, placenta, prostate, skeletal muscle, small intestine, spleen, testis, thyroid, thymus, and uterus tissue. The probes used in the screening were DNA fragments corresponding to AR, ERb, GR and VDR DNA binding domain regions and 250,000 primary clones from the mixed library were screened. After two rounds of screening, in vivo excision was carried out on the purified lZAPII phages which showed positive signals at low stringency condition. Plasmid DNA samples were submitted to sequence directly using M13 forward and reverse primers. Blast search of the sequence information obtained was done using computer program PhredPhrap. Clone gm6 was found to be a novel receptor. DNA sequencing was continued from both 5′ and 3′ ends on nNR7. An intron was identified when sequencing with pgm6y2 (5′-CTTCAATGTCATGACATG-3′; SEQ ID NO; 3). A cDNA fragment flanking the intron region was retrieved via PCR using primer pair of NR7F (5′-CCAAATCTGCCGTGTATGTG-3′; SEQ ID NO; 4) and pgm6xC (5′-GTCAGTGCACTCTCCACGT-3′; SEQ ID NO; 5) followed by double nested primers of pgm6y2 and pgm6xD (5′-TGCAGCTGGTCCACCACGCG-3′; SEQ ID NO; 6). A prominent DNA fragment of ˜1.5 kb was amplified from testis and brain cDNAs. The fragment was purified and subcloned into pCRII vector (Invitrogen, San Diego, Calif.). Automated sequencing was performed on several of the clones. Sequence assembly and analysis were performed with SEQUENCHER™ 3.0 (Gene Codes Corporation, Ann Arbor, Mich.). Ambiguities and/or discrepancies between automated base calling in sequencing reads were visually examined and edited to the correct base call. The final nucleotide sequence encoding nNR7 is shown as set forth in FIGS. 1A-C and as set forth as SEQ ID NO: 1. The complete sequencing of the gm6 clone revealed a 3.1 kb cDNA which codes an authentic novel nuclear receptor. Primer pair pgm6xAR (5′-GGGTATGCTCTGTGACAAG-3′; SEQ ID NO; 7) and pgm6x (5′-AGGCAGGCACTTTCATACC-3′; SEQ ID NO; 8) in the 3′ non-coding region were used to scan the 83 clones of the Stanford radiation hybrid panel (Cox et al., 1990, Science, 250:245-250). The PCR results were scored and submitted to the Stanford Genome Center for linkage analysis. The result indicated that nNR7 was located on chromosome 3. Northern analysis on Clontech blots showed that nNR7 was mainly expressed in human liver at medium to low levels. The liver is the principal site to convert inert forms of D vitamin to active forms which are transported to target tissues, such as intestine, to activate VDR. nNR7 expressed in the liver may be activated by one of the vitamin D metabolites and perform other biological functions of D vitamins. In addition, as discussed herein, nNR7 is involved in regulation a CYP3A4.

### EXAMPLE 2

### Isolation and Characterization of a DNA Molecule Encoding nNR7-1

As disclosed within the specification, the human nNR7 cDNA does not contain a starting Met. Therefore, efforts were made to extend the nNR7 5′-end sequence. Three of the clones from 5′-end RACE (rapid amplification of cDNA end) showed divergence from the original one. One clone contains a starting Met with an inframe stop codon in front. This cDNA clone, nNR7-1, represents a complete open reading frame (ORF) for a human NR7-1 receptor protein.

Oligonucleotide 5′-AAG CCC TTG CAT CCT TCA CA-3′ (SEQ ID NO:19) was paired with primer AP1 5′-CCA TCC TAA TAC GAC TCA CTA TAG GGC-3′ SEQ ID NO:20; Clontech, Palo Alto, Calif.) to amplify nNR7 5′ cDNA ends. The template used was human liver Marathon-Ready™ cDNA from Clontech. DNA product from one round of PCR reaction was gel purified using Qiagen gel extraction kit (Chatsworth, Calif.). The DNA fragment was then subcloned into PCRII™ TA vector (Invitrogen, Carlsbad, Calif.). Plasmid DNA from multiple clones were prepared and submitted for sequencing using the ABI PRISM™ dye terminator cycle sequencing ready reaction kit with AmpliTaq DNA polymerase, FS (Perkin Elmer, Norwalk, Conn.). The sequencing primers used were: TAF 5′-GTA CCG AGC TCG GAT CCA CTA-3′ (SEQ ID NO: 21, TAR 5′-CCG CCA GTG TGA TGG ATA TCT-3′ (SEQ ID NO:22) R7LA 5′-CTC ATC TGC GTT GAC ACT GGG-3′ (SEQ ID NO:23). Sequence assembly and analysis were performed with SEQUENCHER™ 3.0 (Gene Codes Corporation, Ann Arbor, Mich.). Ambiguities and/or discrepancies between automated base calling in sequencing reads were visually examined and edited to the correct base call. Clone 6.2 (refered herein as clone nNR7-1) results in a different open reading frame from the original one but with an in-frame Met and upstream stop codon. The site of divergence between SEQ ID NO:1 and SEQ ID NO:17 (as discused in the Detailed Description of the Invention) is the same as for clone F1 and A2, the other two clones derived from this 5′-RACE experiment. Therefore, it is likely that the cDNA clone which encodes nNR7 (SEQ ID NOs: 1 and 2) is a partially processed cDNA while clone 6.2 (nNR7-1; SEQ ID NOs: 1 and 2) is a completely processed cDNA. As noted in Example 1 for nNR7-1, nNR7-1 is located on chromosome 3, near the 3q13.2 locus. The nNR7-1 clone is expressed in human liver at medium to low levels and within the small intestine. As noted throughout this specification, nNR7-1 will be useful in assays to identify compounds which modulate CYP3A4 expression as well as possible interactions within Vitamin D metobolism.

What is claimed:

1. A purified DNA molecule encoding a human nNR7 protein wherein said protein comprises the amino acid sequence as follows:
SILCTGLFKV DPRGEVGAKN LPPSSPRGPE ANLEVRPKES WNHADFVHCE DTESVPGKPS VNADEEVGGP QICRVCGDKA TGYHFNVMTC EGCKGFFRRA MKRNARLRCP FRKGACEITR KTRRQCQACR LRKCLESGMK KEMIMSDEAV EERRALIKRK KSERTGTQPL GVQGLTEEQR MMIRELMDAQ MKTFDTTFSH FKNFRLPGVL SSGCELPESL QAPSREEAAK WSQVRKDLCS LKVSLQLRGE DGSVWNYKPP ADSGGKEIFS LLPHMADMST YMFKGIISFA KVISYFRDLP IEDQISLLKG AAFELCQLRF NTVFNAETGT WECGRLSYCL EDTAGGFQQL LLEPMLKFHY MLKKLQLHEE EYVLMQAISL FSPDRPGVLQ HRVVDQLQEQ FAITLKSYIE CNRPQPAHRF LFLKIMAMLT ELRSINAQHT QRLLRIQDIH PFATPLMQEL FGITGS, as set forth in three-letter abbreviation in SEQ ID NO: 2.

2. An expression vector for expressing a human nNR7 protein in a recombinant host cell wherein said expression vector comprises a DNA molecule of claim 1.

3. A host cell which expresses a recombinant human nNR7 protein wherein said host cell contains the expression vector of claim 2.

4. A process for expressing a human nNR7 protein in a recombinant host cell, comprising:
(a) transfecting the expression vector of claim 2 into a suitable host cell; and,
(b) culturing the host cells of step (a) under conditions which allow expression of said the human nNR7 protein from said expression vector.

5. A purified DNA molecule encoding a human nNR7 protein wherein said protein consists of the amino acid sequence as follows:
SILCTGLFKV DPRGEVGAKN LPPSSPRGPE ANLEVRPKES WNHADFVHCE DTESVPGKPS VNADEEVGGP QICRVCGDKA TGYHFNVMTC EGCKGFFRRA MKRNARLRCP FRKGACEITR KTRRQCQACR LRKCLESGMK KEMIMSDEAV EERRALIKRK KSERTGTQPL GVQGLTEEQR MMIRELMDAQ MKTFDTTFSH FKNFRLPGVL SSGCELPESL QAPSREEAAK WSQVRKDLCS LKVSLQLRGE DGSVWNYKPP ADSGGKEIFS LLPHMADMST YMFKGIISFA KVISYFRDLP IEDQISLLKG AAFELCQLRF NTVFNAETGT WECGRLSYCL EDTAGGFQQL LLEPMLKFHY MLKKLQLHEE EYVLMQAISL FSPDRPGVLQ HRVVDQLQEQ FAITLKSYIE CNRPQPAHRF LFLKIMAMLT ELRSINAQHT QRLLRIQDIH PFATPLMQEL FGITGS, as set forth in three-letter abbreviation in SEQ ID NO: 2.

6. An expression vector for expressing a human nNR7 protein in a recombinant host cell wherein said expression vector comprises a DNA molecule of claim 5.

7. A host cell which expresses a recombinant human nNR7 protein wherein said host cell contains the expression vector of claim 6.

8. A process for expressing a human nNR7 protein in a recombinant host cell, comprising:
(a) transfecting the expression vector of claim 6 into a suitable host cell; and,
(b) culturing the host cells of step (a) under conditions which allow expression of said the human nNR7 protein from said expression vector.

9. A purified DNA molecule encoding a human nNR7 protein wherein said DNA molecule consists of the nucleotide sequence as set forth in SEQ ID NO: 1, as follows:
TACGCCAAGC TCGAAATTAA CCCTCACTAA AGGGAACAAA AGCTGGAGCT CCACCGCGGT GGCGGCCGCT CTAGAACTAG TGGATCCCCC GGGCTGCAGG AATTCGAATT CTCATAACCT ATGACTAGGA CGGGAAGAGG AAGCACTGCC TTTACTTCAG TGGGAATCTC GGCCTCAGCC TGCAAGCCAA GTGTTCACAG TGAGAAAAGC AAGAGAATAA GCTAATACTC CTGTCCTGAA CAAGGCAGCG GCTCCTTGGT AAAGCTACTC CTTGATCGAT CCTTTGCACC GGATTGTTCA AAGTGGACCC CAGGGGAGAA GTCGGAGCAA AGAACTTACC ACCAAGCAGT CCAAGAGGCC CAGAAGCAAA CCTGGAGGTG AGACCCAAAG AAAGCTGGAA CCATGCTGAC TTTGTACACT GTGAGGACAC AGAGTCTGTT CCTGGAAAGC CCAGTGTCAA CGCAGATGAG GAAGTCGGAG GTCCCCAAAT CTGCCGTGTA TGTGGGGACA AGGCCACTGG CTATCACTTC AATGTCATGA CATGTGAAGG ATGCAAGGGC TTTTTCAGGA GGGCCATGAA ACGCAACGCC CGGCTGAGGT GCCCCTTCCG GAAGGGCGCC TGCGAGATCA CCCGGAAGAC CCGGCGACAG TGCCAGGCCT GCCGCCTGCG CAAGTGCCTG GAGAGCGGCA TGAAGAAGGA GATGATCATG TCCGACGAGG CCGTGGAGGA GAGGCGGGCC TTGATCAAGC GGAAGAAAAG TGAACGGACA GGGACTCAGC CACTGGGAGT GCAGGGGCTG ACAGAGGAGC AGCGGATGAT GATCAGGGAG CTGATGGACG CTCAGATGAA AACCTTTGAC ACTACCTTCT CCCATTTCAA GAATTTCCGG CTGCCAGGGG TGCTTAGCAG TGGCTGCGAG TTGCCAGAGT CTCTGCAGGC CCCATCGAGG GAAGAAGCTG CCAAGTGGAG CCAGGTCCGG AAAGATCTGT GCTCTTTGAA GGTCTCTCTG CAGCTGCGGG GGGAGGATGG CAGTGTCTGG AACTACAAAC CCCCAGCCGA CAGTGGCGGG AAAGAGATCT TCTCCCTGCT GCCCCACATG GCTGACATGT CAACCTACAT GTTCAAAGGC ATCATCAGCT TTGCCAAAGT CATCTCCTAC TTCAGGGACT TGCCCATCGA GGACCAGATC TCCCTGCTGA AGGGGGCCGC TTTCGAGCTG TGTCAACTGA GATTCAACAC AGTGTTCAAC GCGGAGACTG GAACCTGGGA GTGTGGCCGG CTGTCCTACT GCTTGGAAGA CACTGCAGGT GGCTTCCAGC AACTTCTACT GGAGCCCATG CTGAAATTCC ACTACATGCT GAAGAAGCTG CAGCTGCATG AGGAGGAGTA TGTGCTGATG CAGGCCATCT CCCTCTTCTC CCCAGACCGC CCAGGTGTGC TGCAGCACCG CGTGGTGGAC CAGCTGCAGG AGCAATTCGC CATTACTCTG AAGTCCTACA TTGAATGCAA TCGGCCCCAG CCTGCTCATA GGTTCTTGTT CCTGAAGATC ATGGCTATGC TCACCGAGCT CCGCAGCATC AATGCTCAGC ACACCCAGCG GCTGCTGCGC ATCCAGGACA TACACCCCTT TGCTACGCCC CTCATGCAGG AGTTGTTCGG CATCACAGGT AGCTGAGCGG CTGCCCTTGG GTGACACCTC CGAGAGGCAG CCAGACCCAG AGCCCTCTGA GCCGCCACTC CCGGGCCAAG ACAGATGGAC ACTGCCAAGA GCCGACAATG CCCTGCTGGC CTGTCTCCCT AGGGAATTCC TGCTATGACA GCTGGCTAGC ATTCCTCAGG AAGGACATGG GTGCCCCCCA CCCCCAGTTC AGTCTGTAGG GAGTGAAGCC ACAGATTCTT ACGTGGAGAG TGCACTGACC TGTAGGTCAG GACCATCAGA GAGGCAAGGT TGCCCTTTCC TTTTAAAAGG CCCTGTGGTC TGGGGAGAAA TCCCTCAGAT CCCACTAAAG TGTCAAGGTG TGGAAGGGAC CAAGCGACCA AGGATAGGCC ATCTGGGGTC TATGCCCACA TACCCACGTT TGTTCGCTTC CTGAGTCTTT TCATTGCTAC CTCTAATAGT CCTGTCTCCC ACTTCCCACT CGTTCCCCTC CTCTTCCGAG CTGCTTTGTG GGCTCCAGGC CTGTACTCAT CGGCAGGTGC ATGAGTATCT GTGGGAGTCC TCTAGAGAGA TGAGAAGCCA GGAGGCCTGC ACCAAATGTC AGAAGCTTGG CATGACCTCA TTCCGGCCAC ATCATTCTGT GTCTCTGCAT CCATTTGAAC ACATTATTAA GCACCGATAA TAGGTAGCCT GCTGTGGGGT ATACAGCATT GACTCAGATA TAGATCCTGA GCTCACAGAG TTTATAGTTA AAAAAACAAA CAGAAACACA AACAATTTGG ATCAAAAGGA GAAATGATAA GTGACAAAAG CAGCACAAGG AATTTCCCTG TGTGGATGCT GAGCTGTGAT GGCGGGCACT GGGTACCCAA GTGAAGGTTC CCGAGGACAT GAGTCTGTAG GAGCAAGGGC ACAAACTGCA GCTGTGAGTG CGTGTGTGTG ATTTGGTGTA GGTAGGTCTG TTTGCCACTT GATGGGGCCT GGGTTTGTTC CTGGGGCTGG AATGCTGGGT ATGCTCTGTG ACAAGGCTAC GCTGACAATC AGTTAAACAC ACCGGAGAAG AACCATTTAC ATGCACCTTA TATTTCTGTG TACACATCTA TTCTCAAAGC TAAAGGGTAT GAAAGTGCCT GCCTTGTTTA TAGCCACTTG TGAGTAAAAA TTTTTTTGCA TTTTCACAAA TTATACTTTA TATAAGGCAT TCCACACCTA AGAACTAGTT TTGGGAAATG TAGCCCTGGG TTTAATGTCA AATCAAGGCA AAAGGAATTA AATAATGTAC TTTTGGCTAG AGGGGTAAAC TTTTTTGGCC TTTTTCTGGG GAAAATAATG TGGGGGTGTG GGAATTCGAA TTCGATATCA AGCTTATCGA TACCGTCGAC CTCGAGGGGG GGCCCGGTAC CCAATTCGCC CTATAGTGAG TCGTATTACA ATT (SEQ ID NO: 1).

10. A DNA molecule of claim 9 which consists of nucleotide 276 to about nucleotide 1676 of SEQ ID NO: 1.

11. An expression vector for expressing a human nNR7 protein wherein said expression vector comprises a DNA molecule of claim 9.

12. An expression vector for expressing a human nNR7 protein wherein said expression vector comprises a DNA molecule of claim 10.

13. A host cell which expresses a recombinant human nNR7 protein wherein said host cell contains the expression vector of claim 11.

14. A host cell which expresses a recombinant human nNR7 protein wherein said host cell contains the expression vector of claim 12.

15. A process for expressing a human nNR7 protein in a recombinant host cell, comprising:
(a) transfecting the expression vector of claim 11 into a suitable host cell; and,
(b) culturing the host cells of step (a) under conditions which allow expression of said the human nNR7 protein from said

16. A purified human nNR7 protein which comprises the amino acid sequence as set forth in SEQ ID NO:2.

17. A purified human nNR7 protein of claim 16 which consists of the amino acid sequence as set forth in SEQ ID NO:2.

18. A method for determining whether a substance is capable of binding to nNR7 comprising:
(a) providing test cells by transfecting cells with an expression vector that directs the expression of nNR7 in the cells;
(b) exposing the test cells to the substance;
(c) measuring the amount of binding of the substance to nNR7;
(d) comparing the amount of binding of the substance to nNR7 in the test cells with the amount of binding of the substance to control cells that have not been transfected with nNR7.

19. A method of determining whether a substance acts as a modulator of nNR7 activity which comprises:
(a) providing test cells by transfecting cells with a receptor expression vector that directs the expression of nNR7 in the cells;
(b) providing test cells by transfecting the cells of step (a) with a second reporter expression vector that directs expression of a reporter gene under control of a regulatory element which is responsive to nNR7 (b) exposing the test cells to the substance;
(c) measuring the amount of binding of expression of the reporter gene;
(d) comparing the amount of expression of the reporter gene in the test cells with the amount of expression of the reporter gene in control cells that has been transfected with a reporter vector of step (b) but not a receptor vector of step (a).

20. A purified DNA molecule encoding a human nNR7 protein wherein said protein comprises the amino acid sequence as follows:
MTVTRTHHFK EGSLRAPAIP LHSAAAELAS NHPRGPEANL EVRPKESWNH ADFVHCEDTE SVPGKPSVNA DEEVGGPQIC RVCGDKATGY HFNVMTCEGC KGFFRRAMKR NARLRCPFRK GACEITRKTR RQCQACRLRK CLESGMKKEM IMSDEAVEER RALIKRKKSE RTGTQPLGVQ GLTEEQRMMI RELMDAQMKT FDTTFSHFKN FRLPGVLSSG CELPESLQAP SREEAAKWSQ VRKDLCSLKV SLQLRGEDGS VWNYKPPADS GGKEIFSLLP HMADMSTYMF KGIISFAKVI SYFRDLPIED QISLLKGAAF ELCQLRFNTV FNAETGTWEC GRLSYCLEDT AGGFQQLLLE PMLKFHYMLK KLQLHEEEYV LMQAISLFSP DRPGVLQHRV VDQLQEQFAI TLKSYIECNR PQPAHRFLFL KIMAMLTELR SINAQHTQRL LRIQDIHPFA TPLMQELFGI TGS, as set forth in the three-letter abbreviation in SEQ ID NO: 18.

21. An expression vector for expressing a human nNR7-1 protein in a recombinant host cell wherein said expression vector comprises a DNA molecule of claim 20.

22. A host cell which expresses a recombinant human nNR7-1 protein wherein said host cell contains the expression vector of claim 21.

23. A process for expressing a human nNR7-1 protein in a recombinant host cell, comprising:
(a) transfecting the expression vector of claim 21 into a suitable host cell; and,
(b) culturing the host cells of step (a) under conditions which allow expression of said the human nNR7-1 protein from said expression vector.

24. A purified DNA molecule encoding a human nNR7-1 protein wherein said protein consists of the amino acid sequence as follows:
MTVTRTHHFK EGSLRAPAIP LHSAAAELAS NHPRGPEANL EVRPKESWNH ADFVHCEDTE SVPGKPSVNA DEEVGGPQIC RVCGDKATGY HFNVMTCEGC KGFFRRAMKR NARLRCPFRK GACEITRKTR RQCQACRLRK CLESGMKKEM IMSDEAVEER RALIKRKKSE RTGTQPLGVQ GLTEEQRMMI RELMDAQMKT FDTTFSHFKN FRLPGVLSSG CELPESLQAP SREEAAKWSQ VRKDLCSLKV SLQLRGEDGS VWNYKPPADS GGKEIFSLLP HMADMSTYMF KGIISFAKVI SYFRDLPIED QISLLKGAAF ELCQLRFNTV FNAETGTWEC GRLSYCLEDT AGGFQQLLLE PMLKFHYMLK KLQLHEEEYV LMQAISLFSP DRPGVLQHRV VDQLQEQFAI TLKSYIECNR PQPAHRFLFL KIMAMLTELR SINAQHTQRL LRIQDIHPFA TPLMQELFGI TGS, as set forth in the three-letter abbreviation in SEQ ID NO: 18.

25. An expression vector for expressing a human nNR7-1 protein in a recombinant host cell wherein said expression vector comprises a DNA molecule of claim 24.

26. A host cell which expresses a recombinant human nNR7-1 protein wherein said host cell contains the expression vector of claim 25.

27. A process for expressing a human nNR7-1 protein in a recombinant host cell, comprising:
(a) transfecting the expression vector of claim 25 into a suitable host cell; and,
(b) culturing the host cells of step (a) under conditions which allow expression of said the human nNR7-1 protein from said expression vector.

28. A purified DNA molecule encoding a human nNR7-1 protein wherein said DNA molecule consists of the nucleotide sequence as set forth in SEQ ID NO: 17, as follows:
TCCATCCTAA TACGACTCAC TATAGGGCTC GAGCGGCCGC CCGGGCAGGT CTTTTGGCCT GCTGGGTTAG TGCTGGCAGC CCCCTGAGGC CAAGGACAGC AGCATGACAG TCACCAGGAC TCACCACTTC AAGGAGGGGT CCCTCAGAGC ACCTGCCATA CCCCTGCACA GTGCTGCGGC TGAGTTGGCT TCAAACCATC CAAGAGGCCC AGAAGCAAAC CTGGAGGTGA GACCCAAAGA AAGCTGGAAC CATGCTGACT TTGTACACTG TGAGGACACA GAGTCTGTTC CTGGAAAGCC CAGTGTCAAC GCAGATGAGG AAGTCGGAGG TCCCCAAATC TGCCGTGTAT GTGGGGACAA GGCCACTGGC TATCACTTCA ATGTCATGAC ATGTGAAGGA TGCAAGGGCT TTTTCAGGAG GGCCATGAAA CGCAACGCCC GGCTGAGGTG CCCCTTCCGG AAGGGCGCCT GCGAGATCAC CCGGAAGACC CGGCGACAGT GCCAGGCCTG CCGCCTGCGC AAGTGCCTGG AGAGCGGCAT GAAGAAGGAG ATGATCATGT CCGACGAGGC CGTGGAGGAG AGGCGGGCCT TGATCAAGCG GAAGAAAAGT GAACGGACAG GGACTCAGCC ACTGGGAGTG CAGGGGCTGA CAGAGGAGCA GCGGATGATG ATCAGGGAGC TGATGGACGC TCAGATGAAA ACCTTTGACA CTACCTTCTC CCATTTCAAG AATTTCCGGC TGCCAGGGGT GCTTAGCAGT GGCTGCGAGT TGCCAGAGTC TCTGCAGGCC CCATCGAGGG AAGAAGCTGC CAAGTGGAGC CAGGTCCGGA AAGATCTGTG CTCTTTGAAG GTCTCTCTGC AGCTGCGGGG GGAGGATGGC AGTGTCTGGA ACTACAAACC CCCAGCCGAC AGTGGCGGGA AAGAGATCTT CTCCCTGCTG CCCCACATGG CTGACATGTC AACCTACATG TTCAAAGGCA TCATCAGCTT TGCCAAAGTC ATCTCCTACT TCAGGGACTT GCCCATCGAG GACCAGATCT CCCTGCTGAA GGGGGCCGCT TTCGAGCTGT GTCAACTGAG ATTCAACACA GTGTTCAACG CGGAGACTGG AACCTGGGAG TGTGGCCGGC TGTCCTACTG CTTGGAAGAC ACTGCAGGTG GCTTCCAGCA ACTTCTACTG GAGCCCATGC TGAAATTCCA CTACATGCTG AAGAAGCTGC AGCTGCATGA GGAGGAGTAT GTGCTGATGC AGGCCATCTC CCTCTTCTCC CCAGACCGCC CAGGTGTGCT GCAGCACCGC GTGGTGGACC AGCTGCAGGA GCAATTCGCC ATTACTCTGA AGTCCTACAT TGAATGCAAT CGGCCCCAGC CTGCTCATAG GTTCTTGTTC CTGAAGATCA TGGCTATGCT CACCGAGCTC CGCAGCATCA ATGCTCAGCA CACCCAGCGG CTGCTGCGCA TCCAGGACAT ACACCCCTTT GCTACGCCCC TCATGCAGGA GTTGTTCGGC ATCACAGGTA GCTGAGCGGC TGCCCTTGGG TGACACCTCC GAGAGGCAGC CAGACCCAGA GCCCTCTGAG CCGCCACTCC CGGGCCAAGA CAGATGGACA CTGCCAAGAG CCGACAATGC CCTGCTGGCC TGTCTCCCTA GGGAATTCCT GCTATGACAG CTGGCTAGCA TTCCTCAGGA AGGACATGGG TGCCCCCCAC CCCCAGTTCA GTCTGTAGGG AGTGAAGCCA CAGATTCTTA CGTGGAGAGT GCACTGACCT GTAGGTCAGG ACCATCAGAG AGGCAAGGTT GCCCTTTCCT TTTAAAAGGC CCTGTGGTCT GGGGAGAAAT CCCTCAGATC CCACTAAAGT GTCAAGGTGT GGAAGGGACC AAGCGACCAA GGATAGGCCA TCTGGGGTCT ATGCCCACAT ACCCACGTTT GTTCGCTTCC TGAGTCTTTT CATTGCTACC TCTAATAGTC CTGTCTCCCA CTTCCCACTC GTTCCCCTCC TCTTCCGAGC TGCTTTGTGG GCTCCAGGCC TGTACTCATC GGCAGGTGCA TGAGTATCTG TGGGAGTCCT CTAGAGAGAT GAGAAGCCAG GAGGCCTGCA CCAAATGTCA GAAGCTTGGC ATGACCTCAT TCCGGCCACA TCATTCTGTG TCTCTGCATC CATTTGAACA CATTATTAAG CACCGATAAT AGGTAGCCTG CTGTGGGGTA TACAGCATTG ACTCAGATAT AGATCCTGAG CTCACAGAGT TTATAGTTAA AAAAACAAAC AGAAACACAA ACAATTTGGA TCAAAAGGAG AAATGATAAG TGACAAAAGC AGCACAAGGA ATTTCCCTGT GTGGATGCTG AGCTGTGATG GCGGGCACTG GGTACCCAAG TGAAGGTTCC CGAGGACATG AGTCTGTAGG AGCAAGGGCA CAAACTGCAG CTGTGAGTGC GTGTGTGTGA TTTGGTGTAG GTAGGTCTGT TTGCCACTTG ATGGGGCCTG GGTTTGTTCC TGGGGCTGGA ATGCTGGGTA TGCTCTGTGA CAAGGCTACG CTGACAATCA GTTAAACACA CCGGAGAAGA ACCATTTACA TGCACCTTAT ATTTCTGTGT ACACATCTAT TCTCAAAGCT AAAGGGTATG AAAGTGCCTG CCTTGTTTAT AGCCACTTGT GAGTAAAAAT TTTTTTGCAT TTTCACAAAT TATACTTTAT ATAAGGCATT CCACACCTAA GAACTAGTTT TGGGAAATGT AGCCCTGGGT TTAATGTCAA ATCAAGGCAA AAGGAATTAA ATAATGTACT TTTGGCTAGA GGGGTAAACT TTTTTGGCCT TTTTCTGGGG AAAATAATGT GGGGGTGTGG (SEQ ID NO: 17).

29. A DNA molecule of claim 28 which consists of nucleotide 104 to about nucleotide 1525 of SEQ ID NO: 17.

30. An expression vector for expressing a human nNR7-1 protein wherein said expression vector comprises a DNA molecule of claim 28.

31. An expression vector for expressing a human nNR7-1 protein wherein said expression vector comprises a DNA molecule of claim 29.

32. A host cell which expresses a recombinant human nNR7-1 protein wherein said host cell contains the expression vector of claim 30.

33. A host cell which expresses a recombinant human nNR7-1 protein wherein said host cell contains the expression vector of claim 31.

34. A process for expressing a human nNR7 protein in a recombinant host cell, comprising:
(a) transfecting the expression vector of claim 29 into a suitable host cell; and,
(b) culturing the host cells of step (a) under conditions which allow expression of said the human nNR7-1 protein from said expression vector.

35. A purified human nNR7-1 protein which comprises the amino acid sequence as set forth in SEQ ID NO:18.

36. A purified human nNR7-1 protein of claim 35 which consists of the amino acid sequence as set forth in SEQ ID NO:18.

37. A method for determining whether a substance is capable of binding to nNR7-1 comprising:
(a) providing test cells by transfecting cells with an expression vector that directs the expression of nNR7-1 in the cells;
(b) exposing the test cells to the substance;
(c) measuring the amount of binding of the substance to nNR7-1;
(d) comparing the amount of binding of the substance to nNR7-1 in the test cells with the amount of binding of the substance to control cells that have not been transfected with nNR7-1.

38. A method of determining whether a substance acts as a modulator of nNR7-1 activity which comprises:
(a) providing test cells by transfecting cells with a receptor expression vector that directs the expression of nNR7-1 in the cells;
(b) providing test cells by transfecting the cells of step (a) with a second reporter expression vector that directs expression of a reporter gene under control of a regulatory element which is responsive to nNR7-1 (b) exposing the test cells to the substance;
(c) measuring the amount of binding of expression of the reporter gene;
(d) comparing the amount of expression of the reporter gene in the test cells with the amount of expression of the reporter gene in control cells that has been transfected with a reporter vector of step (b) but not a receptor vector of step (a).


SEQUENCE LISTING (24 sequences)

SEQ ID NO: 1 - DNA, 3093 bp, Homo sapien (human)
tacgccaagctcgaaattaaccctcactaaagggaacaaaagctggagctccaccgcggtggcggccgctctagaactagtggatcccccgggctgcaggaattcgaattctcataacctatgactaggacgggaagaggaagcactgcctttacttcagtgggaatctcggcctcagcctgcaagccaagtgttcacagtgagaaaagcaagagaataagctaatactcctgtcctgaacaaggcagcggctccttggtaaagctactccttgatcgatcctttgcaccggattgttcaaagtggaccccaggggagaagtcggagcaaagaacttaccaccaagcagtccaagaggcccagaagcaaacctggaggtgagacccaaagaaagctggaaccatgctgactttgtacactgtgaggacacagagtctgttcctggaaagcccagtgtcaacgcagatgaggaagtcggaggtccccaaatctgccgtgtatgtggggacaaggccactggctatcacttcaatgtcatgacatgtgaaggatgcaagggctttttcaggagggccatgaaacgcaacgcccggctgaggtgccccttccggaagggcgcctgcgagatcacccggaagacccggcgacagtgccaggcctgccgcctgcgcaagtgcctggagagcggcatgaagaaggagatgatcatgtccgacgaggccgtggaggagaggcgggccttgatcaagcggaagaaaagtgaacggacagggactcagccactgggagtgcaggggctgacagaggagcagcggatgatgatcagggagctgatggacgctcagatgaaaacctttgacactaccttctcccatttcaagaatttccggctgccaggggtgcttagcagtggctgcgagttgccagagtctctgcaggccccatcgagggaagaagctgccaagtggagccaggtccggaaagatctgtgctctttgaaggtctctctgcagctgcggggggaggatggcagtgtctggaactacaaacccccagccgacagtggcgggaaagagatcttctccctgctgccccacatggctgacatgtcaacctacatgttcaaaggcatcatcagctttgccaaagtcatctcctacttcagggacttgcccatcgaggaccagatctccctgctgaagggggccgctttcgagctgtgtcaactgagattcaacacagtgttcaacgcggagactggaacctgggagtgtggccggctgtcctactgcttggaagacactgcaggtggcttccagcaacttctactggagcccatgctgaaattccactacatgctgaagaagctgcagctgcatgaggaggagtatgtgctgatgcaggccatctccctcttctccccagaccgcccaggtgtgctgcagcaccgcgtggtggaccagctgcaggagcaattcgccattactctgaagtcctacattgaatgcaatcggccccagcctgctcataggttcttgttcctgaagatcatggctatgctcaccgagctccgcagcatcaatgctcagcacacccagcggctgctgcgcatccaggacatacacccctttgctacgcccctcatgcaggagttgttcggcatcacaggtagctgagcggctgcccttgggtgacacctccgagaggcagccagacccagagccctctgagccgccactcccgggccaagacagatggacactgccaagagccgacaatgccctgctggcctgtctccctagggaattcctgctatgacagctggctagcattcctcaggaaggacatgggtgccccccacccccagttcagtctgtagggagtgaagccacagattcttacgtggagagtgcactgacctgtaggtcaggaccatcagagaggcaaggttgccctttccttttaaaaggccctgtggtctggggagaaatccctcagatcccactaaagtgtcaaggtgtggaagggaccaagcgaccaaggataggccatctggggtctatgcccacatacccacgtttgttcgcttcctgagtcttttcattgctacctctaatagtcctgtctcccacttcccactcgttcccctcctcttccgagctgctttgtgggctccaggcctgtactcatcggcaggtgcatgagtatctgtgggagtcctctagagagatgagaagccaggaggcctgcaccaaatgtcagaagcttggcatgacctcattccggccacatcattctgtgtctctgcatccatttgaacacattattaagcaccgataataggtagcctgctgtggggtatacagcattgactcagatatagatcctgagctcacagagtttatagttaaaaaaacaaacagaaacacaaacaatttggatcaaaaggagaaatgataagtgacaaaagcagcacaaggaatttccctgtgtggatgctgagctgtgatggcgggcactgggtacccaagtgaaggttcccgaggacatgagtctgtaggagcaagggcacaaactgcagctgtgagtgcgtgtgtgtgatttggtgtaggtaggtctgtttgccacttgatggggcctgggtttgttcctggggctggaatgctgggtatgctctgtgacaaggctacgctgacaatcagttaaacacaccggagaagaaccatttacatgcaccttatatttctgtgtacacatctattctcaaagctaaagggtatgaaagtgcctgccttgtttatagccacttgtgagtaaaaatttttttgcattttcacaaattatactttatataaggcattccacacctaagaactagttttgggaaatgtagccctgggtttaatgtcaaatcaaggcaaaaggaattaaataatgtacttttggctagaggggtaaacttttttggcctttttctggggaaaataatgtgggggtgtgggaattcgaattcgatatcaagcttatcgataccgtcgacctcgagggggggcccggtacccaattcgccctatagtgagtcgtattacaatt

SEQ ID NO: 2 - protein, 466 aa, Homo sapien (human)
SILCTGLFKVDPRGEVGAKNLPPSSPRGPEANLEVRPKESWNHADFVHCEDTESVPGKPSVNADEEVGGPQICRVCGDKATGYHFNVMTCEGCKGFFRRAMKRNARLRCPFRKGACEITRKTRRQCQACRLRKCLESGMKKEMIMSDEAVEERRALIKRKKSERTGTQPLGVQGLTEEQRMMIRELMDAQMKTFDTTFSHFKNFRLPGVLSSGCELPESLQAPSREEAAKWSQVRKDLCSLKVSLQLRGEDGSVWNYKPPADSGGKEIFSLLPHMADMSTYMFKGIISFAKVISYFRDLPIEDQISLLKGAAFELCQLRFNTVFNAETGTWECGRLSYCLEDTAGGFQQLLLEPMLKFHYMLKKLQLHEEEYVLMQAISLFSPDRPGVLQHRVVDQLQEQFAITLKSYIECNRPQPAHRFLFLKIMAMLTELRSINAQHTQRLLRIQDIHPFATPLMQELFGITGS

SEQ ID NO: 3 - DNA, 18 bp, Artificial Sequence
  Oligonucleotide
cttcaatgtcatgacatg

SEQ ID NO: 4 - DNA, 20 bp, Artificial Sequence
  Oligonucleotide
ccaaatctgccgtgtatgtg

SEQ ID NO: 5 - DNA, 19 bp, Artificial Sequence
  Oligonucleotide
gtcagtgcactctccacgt

SEQ ID NO: 6 - DNA, 20 bp, Artificial Sequence
  Oligonucleotide
tgcagctggtccaccacgcg

SEQ ID NO: 7 - DNA, 19 bp, Artificial Sequence
  Oligonucleotide
gggtatgctctgtgacaag

SEQ ID NO: 8 - DNA, 19 bp, Artificial Sequence
  Oligonucleotide
aggcaggcactttcatacc

SEQ ID NO: 9 - DNA, 20 bp, Artificial Sequence
  Oligonucleotide
tttcgagcttccaggttcat

SEQ ID NO: 10 - DNA, 20 bp, Artificial Sequence
  Oligonucleotide
ctcccaaactctgcctggtg

SEQ ID NO: 11 - DNA, 20 bp, Artificial Sequence
  Oligonucleotide
cgggagccacacttcaccat

SEQ ID NO: 12 - DNA, 20 bp, Artificial Sequence
  Oligonucleotide
gctcacttctgcgctgtctg

SEQ ID NO: 13 - DNA, 20 bp, Artificial Sequence
  Oligonucleotide
ttccgggctcccagagtcat

SEQ ID NO: 14 - DNA, 20 bp, Artificial Sequence
  Oligonucleotide
cagaagacctgcctgatctg

SEQ ID NO: 15 - DNA, 20 bp, Artificial Sequence
  Oligonucleotide
gaaatgaactccttcatcat

SEQ ID NO: 16 - DNA, 20 bp, Artificial Sequence
  Oligonucleotide
ccggatctgtggggtgtgtg

SEQ ID NO: 17 - DNA, 2850 bp, Homo sapien (human)
tccatcctaatacgactcactatagggctcgagcggccgcccgggcaggtcttttggcctgctgggttagtgctggcagccccctgaggccaaggacagcagcatgacagtcaccaggactcaccacttcaaggaggggtccctcagagcacctgccatacccctgcacagtgctgcggctgagttggcttcaaaccatccaagaggcccagaagcaaacctggaggtgagacccaaagaaagctggaaccatgctgactttgtacactgtgaggacacagagtctgttcctggaaagcccagtgtcaacgcagatgaggaagtcggaggtccccaaatctgccgtgtatgtggggacaaggccactggctatcacttcaatgtcatgacatgtgaaggatgcaagggctttttcaggagggccatgaaacgcaacgcccggctgaggtgccccttccggaagggcgcctgcgagatcacccggaagacccggcgacagtgccaggcctgccgcctgcgcaagtgcctggagagcggcatgaagaaggagatgatcatgtccgacgaggccgtggaggagaggcgggccttgatcaagcggaagaaaagtgaacggacagggactcagccactgggagtgcaggggctgacagaggagcagcggatgatgatcagggagctgatggacgctcagatgaaaacctttgacactaccttctcccatttcaagaatttccggctgccaggggtgcttagcagtggctgcgagttgccagagtctctgcaggccccatcgagggaagaagctgccaagtggagccaggtccggaaagatctgtgctctttgaaggtctctctgcagctgcggggggaggatggcagtgtctggaactacaaacccccagccgacagtggcgggaaagagatcttctccctgctgccccacatggctgacatgtcaacctacatgttcaaaggcatcatcagctttgccaaagtcatctcctacttcagggacttgcccatcgaggaccagatctccctgctgaagggggccgctttcgagctgtgtcaactgagattcaacacagtgttcaacgcggagactggaacctgggagtgtggccggctgtcctactgcttggaagacactgcaggtggcttccagcaacttctactggagcccatgctgaaattccactacatgctgaagaagctgcagctgcatgaggaggagtatgtgctgatgcaggccatctccctcttctccccagaccgcccaggtgtgctgcagcaccgcgtggtggaccagctgcaggagcaattcgccattactctgaagtcctacattgaatgcaatcggccccagcctgctcataggttcttgttcctgaagatcatggctatgctcaccgagctccgcagcatcaatgctcagcacacccagcggctgctgcgcatccaggacatacacccctttgctacgcccctcatgcaggagttgttcggcatcacaggtagctgagcggctgcccttgggtgacacctccgagaggcagccagacccagagccctctgagccgccactcccgggccaagacagatggacactgccaagagccgacaatgccctgctggcctgtctccctagggaattcctgctatgacagctggctagcattcctcaggaaggacatgggtgccccccacccccagttcagtctgtagggagtgaagccacagattcttacgtggagagtgcactgacctgtaggtcaggaccatcagagaggcaaggttgccctttccttttaaaaggccctgtggtctggggagaaatccctcagatcccactaaagtgtcaaggtgtggaagggaccaagcgaccaaggataggccatctggggtctatgcccacatacccacgtttgttcgcttcctgagtcttttcattgctacctctaatagtcctgtctcccacttcccactcgttcccctcctcttccgagctgctttgtgggctccaggcctgtactcatcggcaggtgcatgagtatctgtgggagtcctctagagagatgagaagccaggaggcctgcaccaaatgtcagaagcttggcatgacctcattccggccacatcattctgtgtctctgcatccatttgaacacattattaagcaccgataataggtagcctgctgtggggtatacagcattgactcagatatagatcctgagctcacagagtttatagttaaaaaaacaaacagaaacacaaacaatttggatcaaaaggagaaatgataagtgacaaaagcagcacaaggaatttccctgtgtggatgctgagctgtgatggcgggcactgggtacccaagtgaaggttcccgaggacatgagtctgtaggagcaagggcacaaactgcagctgtgagtgcgtgtgtgtgatttggtgtaggtaggtctgtttgccacttgatggggcctgggtttgttcctggggctggaatgctgggtatgctctgtgacaaggctacgctgacaatcagttaaacacaccggagaagaaccatttacatgcaccttatatttctgtgtacacatctattctcaaagctaaagggtatgaaagtgcctgccttgtttatagccacttgtgagtaaaaatttttttgcattttcacaaattatactttatataaggcattccacacctaagaactagttttgggaaatgtagccctgggtttaatgtcaaatcaaggcaaaaggaattaaataatgtacttttggctagaggggtaaacttttttggcctttttctggggaaaataatgtgggggtgtgg

SEQ ID NO: 18 - protein, 473 aa, Homo sapien (human)
MTVTRTHHFKEGSLRAPAIPLHSAAAELASNHPRGPEANLEVRPKESWNHADFVHCEDTESVPGKPSVNADEEVGGPQICRVCGDKATGYHFNVMTCEGCKGFFRRAMKRNARLRCPFRKGACEITRKTRRQCQACRLRKCLESGMKKEMIMSDEAVEERRALIKRKKSERTGTQPLGVQGLTEEQRMMIRELMDAQMKTFDTTFSHFKNFRLPGVLSSGCELPESLQAPSREEAAKWSQVRKDLCSLKVSLQLRGEDGSVWNYKPPADSGGKEIFSLLPHMADMSTYMFKGIISFAKVISYFRDLPIEDQISLLKGAAFELCQLRFNTVFNAETGTWECGRLSYCLEDTAGGFQQLLLEPMLKFHYMLKKLQLHEEEYVLMQAISLFSPDRPGVLQHRVVDQLQEQFAITLKSYIECNRPQPAHRFLFLKIMAMLTELRSINAQHTQRLLRIQDIHPFATPLMQELFGITGS

SEQ ID NO: 19 - DNA, 20 bp, Artificial Sequence
  Oligonucleotide
aagcccttgcatccttcaca

SEQ ID NO: 20 - DNA, 27 bp, Artificial Sequence
  Oligonucleotide
ccatcctaatacgactcactatagggc

SEQ ID NO: 21 - DNA, 21 bp, Artificial Sequence
  Oligonucleotide
gtaccgagctcggatccacta

SEQ ID NO: 22 - DNA, 21 bp, Artificial Sequence
  Oligonucleotide
ccgccagtgtgatggatatct

SEQ ID NO: 23 - DNA, 21 bp, Artificial Sequence
  Oligonucleotide
ctcatctgcgttgacactggg

SEQ ID NO: 24 - DNA, 18 bp, Artificial Sequence
  Oligonucleotide
tgaactcaaaggaggtca